SEQUENCE LISTING (14 sequences)

SEQ ID NO: 1 - DNA, 1178 bp, Helianthus annus
  CDS (3)...(1178)
tcttcgcctaccccggcggcacgtcaatggagatccaccaagctctcacgcgctcaagcactatccgcaatgtgctcccccgtcacgaacagggcggcgtgttcgccgccgaaggctacgcgcgcgcctccggtcttcccggcgtgtgtatcgccacttccggtcccggagctacgaacctagttagtggtcttgctgacgcgctgttagacagtgtccccatggtggcaatcaccggtcaagttccccggagaatgatcggaaccgatgcgtttcaagaaaccccaattgttgaggtaacacgttcgatcactaaacataattatcttgtgttggatgttgaggatattcccagaattgttcgtgaggctttttatcttgcgagttcgggtcgacccggcccggttttgatagatgtaccgaaagatatacagcaacagttagtggtgccgaaatgggatgaaccgatgaggttaccgggttatttgtctagaatgccgaagcctcaatatgatgggcatttggaacagattgttaggttggtgggggaagcgaagaggccggttttgtatgtgggtggtgggtgtttgaattcggatgatgagttgaggcggtttgtggagcttacggggattccggttgcgagtactttgatggggctcggagcgtaccctgcttcgagtgatttgtcgcttcatatgcttgggatgcatggtacggtttatgcgaattatgcggttgataagagtgatttgttgcttgcgtttggggtgcggtttgatgatcgtgtgacggggaagcttgaggcgtttgctagtagggcgaagattgttcatattgatattgatcctgctgaaattgggaagaataagcagcctcatgtgtcgatttgtggtgatattaaggtcgcgttacagggtttgaacaagattttggaggaaaagaattcggtgactaatcttgatttttcgacctggagaaaggaattggatgaacaaaaaatgaagttcccgttgagctttaaaacgtttggcgaagcgattcctccacagtatgctattcaagttcttgatgagttaacgggcgggaatgcaattattagcaccggtgtcgggcaacatcagatgtgggctgctcagttttacaaatacaacaaacctagacaatggctgacgtcg

SEQ ID NO: 2 - protein, 392 aa, Helianthus annus
FAYPGGTSMEIHQALTRSSTIRNVLPRHEQGGVFAAEGYARASGLPGVCIATSGPGATNLVSGLADALLDSVPMVAITGQVPRRMIGTDAFQETPIVEVTRSITKHNYLVLDVEDIPRIVREAFYLASSGRPGPVLIDVPKDIQQQLVVPKWDEPMRLPGYLSRMPKPQYDGHLEQIVRLVGEAKRPVLYVGGGCLNSDDELRRFVELTGIPVASTLMGLGAYPASSDLSLHMLGMHGTVYANYAVDKSDLLLAFGVRFDDRVTGKLEAFASRAKIVHIDIDPAEIGKNKQPHVSICGDIKVALQGLNKILEEKNSVTNLDFSTWRKELDEQKMKFPLSFKTFGEAIPPQYAIQVLDELTGGNAIISTGVGQHQMWAAQFYKYNKPRQWLTS

SEQ ID NO: 3 - DNA, 1178 bp, Helianthus annus
  CDS (3)...(1178)
tcttcgcctaccccggcggcgcgtcaatggagatccaccaagctctcacgcgctcaagcactatccgcaatgtgctcccccgtcacgaacagggcggcgtgttcgccgccgaaggctacgcgcgcgcctccggtcttcccggcgtgtgtatcgccacttccggtcccggagctacgaacctagttagtggtcttgctgacgcgctgttagacagtgtccccatggtggcaatcaccggtcaagttccccggagaatgatcggaaccgatgcgtttcaagaaaccccaattgttgaggtaacacgttcgatcactaaacataattatcttgtgttggatgttgaggatattcccagaattgttcgtgaggctttttatcttgcgagttcgggtcgacccggcccggttttgatagatgtaccgaaagatatacagcaacagttagtggtgccgaaatgggatgaaccgatgaggttaccgggttatttgtctagaatgccgaagcctcaatatgatgggcatttggaacagattgttaggttggtgggggaagcgaagaggccggttttgtatgtgggtggtgggtgtttgaattcggatgatgagttgaggcggtttgtggagcttacggggattccggttgcgagtactttgatggggctcggagcgtaccctgcttcgagtgatttgtcgcttcatatgcttgggatgcatggtacggtttatgcgaattatgcggttgataagagtgatttgttgcttgcgtttggggtgcggtttgatgatcgtgtgacggggaagcttgaggcgtttgctagtagggcgaagattgttcatattgatattgatcctgctgaaattgggaagaataagcagcctcatgtgtcgatttgtggtgatattaaggtcgcgttacagggtttgaacaagattttggaggaaaagaattcggtgactaatcttgatttttcgacctggagaaaggaattggatgaacaaaaaatgaagttcccgttgagctttaaaacgtttggcgaagcgattcctccacagtatgctattcaagttcttgatgagttaacgggcgggaatgcaattattagcaccggtgtcgggcaacatcagatgtgggctgctcagttttacaaatacaacaaacctagacaatggctgacgtcg

SEQ ID NO: 4 - protein, 392 aa, Helianthus annus
FAYPGGASMEIHQALTRSSTIRNVLPRHEQGGVFAAEGYARASGLPGVCIATSGPGATNLVSGLADALLDSVPMVAITGQVPRRMIGTDAFQETPIVEVTRSITKHNYLVLDVEDIPRIVREAFYLASSGRPGPVLIDVPKDIQQQLVVPKWDEPMRLPGYLSRMPKPQYDGHLEQIVRLVGEAKRPVLYVGGGCLNSDDELRRFVELTGIPVASTLMGLGAYPASSDLSLHMLGMHGTVYANYAVDKSDLLLAFGVRFDDRVTGKLEAFASRAKIVHIDIDPAEIGKNKQPHVSICGDIKVALQGLNKILEEKNSVTNLDFSTWRKELDEQKMKFPLSFKTFGEAIPPQYAIQVLDELTGGNAIISTGVGQHQMWAAQFYKYNKPRQWLTS

SEQ ID NO: 5 - DNA, 2156 bp, Xanthium sp.
  CDS (111)...(2057)
  misc_feature (0)...(0): GenBank Accession No. U16280
gaacaacagccacatgtttctggaccatcgtcgttcacacctattttaatcagataaacaaagtacaaacataacataacataaccctagtacataacacacattcaacaatggcggccatccctcatacaaacccttccatcaccaccaaaccaccctcatctccaccacgtcccaccttcctcgcccgtttcacattcccaataacctccacttcccataaacgacaccgtctccacatctccaacgtcctctccgactccaaacccaccatcacccattcaccattaccaaccaaatcatttatctcccgttacgctccagaccaaccaagaaaaggcgctgatgttctcgtcgaagctctggaacgtgaaggcgttacagacgtcttcgcttacccaggtggtgcctccatggagatccaccaagctctcacgcgctcaaccaccatccgcaacgttctcccacgtcacgaacagggcggcgtctttgctgccgaaggctacgcacgtgcctccggtcttcccggcgtctgtattgcaacctctggtcctggagctacgaacctagtaagtggtcttgctgatgctttattagacagtgttccaatggttgctattactggtcaagttcccaggagaatgattggaacagatgcgtttcaagaaacccctattgttgaggtaacacgttccattactaagcataattatttagttttggatgtcgaggatattcccaggattgttagggaagctttttatcttgcgtcttctggtcgacccggaccggttttaattgatgtacctaaggatatacagcagcagttggtagtgcctaaatgggatgagcctattaggttacctgggtatttgtctaggttccctaaaacggagaataatgggcagttggaacagattgttaggttggtgagtgaggccaagaggccggttttgtatgtggggggtgggtgtttgaattcgggagatgagttgaggcggtttgtggagcttacggggataccggttgcgagtacgttgatggggcttggagcgtaccctgcttctagtgatttgtcgctgcatatgcttgggatgcatgggaccgtttatgcgaattatgcggttgataagagtgatttgttgcttgcgtttggggtaaggtttgatgaccgtgtgacggggaagcttgaggcttttgctagcagagctaagattgttcatattgatattgattctgcggaaattgggaagaataagcagcctcatgtgtcgatttgtggtgatattaaggtcgcgttacagggtctgaacaagattttggaggtaaagaattcggtgactaatcttgatttctcgaactggaggaaggaattggatgagcaaaaggttaagtatccgttgagttttaaaacatttggcgaagctattcctccgcagtatgccattcaagtgcttgatgagttaacgggtgggaatgcgattattagcactggggtcgggcagcatcagatgtgggctgctcagttttacaaatacaacaagcctagacaatggctgacgtcaggtggactaggggcgatgggttttgggttgcccgctgctatcggggccgctgttgcaagacctgatgcggtagtagttgatatcgatggtgatggaagctttataatgaacgttcaagagttagccacaatccgtgttgaaaatcttcctgttaagattttgttacttaacaatcagcatttgggtatggtggttcagtgggaggatcggttttacaaggcgaatcgggctcatacctacttaggaaatccgtcaaaagagtctgaaatattccctaacatgttgaagtttgctgaagcgtgtgatatcccagctgcccgagtgacccggaaggcagatctacgagcagctattcagaagatgttggatacaccggggccttacttgttggatgtgatcgtgccccatcaagaacatgtgttgcccatgatcccggctggtggaggtttcatggatgtgatcaccgaaggcgacggcagaatgaaatattgagcttcaatgtcacatatagtgtgttctgtaagcagtttgtcggttatgaagttaaatgttttgttgtgtaatttcgttcctggttaaaaaatcaagctt

SEQ ID NO: 6 - protein, 648 aa, Xanthium sp.
MAAIPHTNPSITTKPPSSPPRPTFLARFTFPITSTSHKRHRLHISNVLSDSKPTITHSPLPTKSFISRYAPDQPRKGADVLVEALEREGVTDVFAYPGGASMEIHQALTRSTTIRNVLPRHEQGGVFAAEGYARASGLPGVCIATSGPGATNLVSGLADALLDSVPMVAITGQVPRRMIGTDAFQETPIVEVTRSITKHNYLVLDVEDIPRIVREAFYLASSGRPGPVLIDVPKDIQQQLVVPKWDEPIRLPGYLSRFPKTENNGQLEQIVRLVSEAKRPVLYVGGGCLNSGDELRRFVELTGIPVASTLMGLGAYPASSDLSLHMLGMHGTVYANYAVDKSDLLLAFGVRFDDRVTGKLEAFASRAKIVHIDIDSAEIGKNKQPHVSICGDIKVALQGLNKILEVKNSVTNLDFSNWRKELDEQKVKYPLSFKTFGEAIPPQYAIQVLDELTGGNAIISTGVGQHQMWAAQFYKYNKPRQWLTSGGLGAMGFGLPAAIGAAVARPDAVVVDIDGDGSFIMNVQELATIRVENLPVKILLLNNQHLGMVVQWEDRFYKANRAHTYLGNPSKESEIFPNMLKFAEACDIPAARVTRKADLRAAIQKMLDTPGPYLLDVIVPHQEHVLPMIPAGGGFMDVITEGDGRMKY

SEQ ID NO: 7 - DNA, 19 bp, Artificial Sequence
  Primer HA1U409
cagacgtgttggtggaagc

SEQ ID NO: 8 - DNA, 21 bp, Artificial Sequence
  Primer HA1L1379
ctgtaacgcgaccttaatatc

SEQ ID NO: 9 - DNA, 22 bp, Artificial Sequence
  Primer HA1U1313
tgctgaaattgggaagaataag

SEQ ID NO: 10 - DNA, 19 bp, Artificial Sequence
  Primer HA1L2131
tttcgttctgccatcaccc

SEQ ID NO: 11 - DNA, 1968 bp, Helianthus annus
  CDS (1)...(1965)
  misc_feature (0)...(0): GenBank Accession No. AY541451
atggcggctcctcccaacccttccatctccttcaaaccaccgtcacccgccgccgcactgccaccacgctccgccttcctcccccgtttcgcattacccatcacttccactacccaaaaacgacaccgtcttcacatctccaatgttctctccgactccaaatccaccaccaccaccaccaccaccactcaacgaccgttaccggtgcagccttttgtctcccgttacgcgccagatcaaccgagaaaaggcgcagacgtgttggtggaagctctggaacgggaaggtgtcaccgacgtcttcgcctaccccggcggcgcgtcaatggagatccaccaagctctcacgcgctcaagcactatccgcaatgtgctcccccgtcacgaacagggcggcgtgttcgccgccgaaggctacgcgcgcgcctccggtcttcccggcgtgtgtatcgccacttccggtcccggagctacgaacctagttagtggtcttgctgacgcgctgttagacagtgtccccatggtggcaatcaccggtcaagttccccggagaatgatcggaaccgatgcgtttcaagaaaccccaattgttgaggtaacacgttcgatcactaaacataattatcttgtgttggatgttgaggatattcccagaattgttcgtgaggctttttatcttgcgagttcgggtcgacccggcccggttttgatagatgtaccgaaagatatacagcaacagttagtggtgccgaaatgggatgaaccgatgaggttaccgggttatttgtctagaatgccgaagcctcaatatgatgggcatttggaacagattgttaggttggtgggggaagcgaagaggccggttttgtatgtgggtggtgggtgtttgaattcggatgatgagttgaggcggtttgtggagcttacggggattccggttgcgagtactttgatggggctcggagcgtaccctgcttcgagtgatttgtcgcttcatatgcttgggatgcatggtacggtttatgcgaattatgcggttgataagagtgatttgttgcttgcgtttggggtgcggtttgatgatcgtgtgacggggaagcttgaggcgtttgctagtagggcgaagattgttcatattgatattgatcctgctgaaattgggaagaataagcagcctcatgtgtcgatttgtggtgatattaaggtcgcgttacagggtttgaacaagattttggaggaaaagaattcggtgactaatcttgatttttcgacctggagaaaggaattggatgaacaaaaaatgaagttcccgttgagctttaaaacgtttggcgaagcgattcctccacagtatgctattcaagttcttgatgagttaacgggcgggaatgcaattattagcaccggtgtcgggcaacatcagatgtgggctgctcagttttacaaatacaacaaacctagacaatggctgacgtcgggcgggctaggggcaatgggtttcggcctgcccgctgctatcggggcggccgttgcaagacctgatgcggtagtagttgacatcgacggtgacggaagctttatgatgaatgttcaagagttagccacaatccgtgttgaaaatctgccggttaagattttattacttaacaaccagcatttgggtatggtggttcagtgggaggatcggttttacaaggcgaatcgggctcatacctacttaggaaacccgtcaaaagagtcggaaatattccctaacatggtgaagtttgctgaagcctgtgatatcccggctgctcgagtgacccaaaaggcggatctacgagcagctattcagaagatgttggatacacccgggccttacttgttggatgtgattgtgccgcatcaagaacacgtgttgcccatgatcccggctggcggaggtttctcggatgtgatcaccgagggtgatggcagaacgaaatattga

SEQ ID NO: 12 - protein, 655 aa, Helianthus annus
MAAPPNPSISFKPPSPAAALPPRSAFLPRFALPITSTTQKRHRLHISNVLSDSKSTTTTTTTTQRPLPVQPFVSRYAPDQPRKGADVLVEALEREGVTDVFAYPGGASMEIHQALTRSSTIRNVLPRHEQGGVFAAEGYARASGLPGVCIATSGPGATNLVSGLADALLDSVPMVAITGQVPRRMIGTDAFQETPIVEVTRSITKHNYLVLDVEDIPRIVREAFYLASSGRPGPVLIDVPKDIQQQLVVPKWDEPMRLPGYLSRMPKPQYDGHLEQIVRLVGEAKRPVLYVGGGCLNSDDELRRFVELTGIPVASTLMGLGAYPASSDLSLHMLGMHGTVYANYAVDKSDLLLAFGVRFDDRVTGKLEAFASRAKIVHIDIDPAEIGKNKQPHVSICGDIKVALQGLNKILEEKNSVTNLDFSTWRKELDEQKMKFPLSFKTFGEAIPPQYAIQVLDELTGGNAIISTGVGQHQMWAAQFYKYNKPRQWLTSGGLGAMGFGLPAAIGAAVARPDAVVVDIDGDGSFMMNVQELATIRVENLPVKILLLNNQHLGMVVQWEDRFYKANRAHTYLGNPSKESEIFPNMVKFAEACDIPAARVTQKADLRAAIQKMLDTPGPYLLDVIVPHQEHVLPMIPAGGGFSDVITEGDGRTKY

SEQ ID NO: 13 - DNA, 2013 bp, Arabidopsis thaliana
  CDS (1)...(2013)
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggacggtgtatgcgaattacgctgtggagcatagtgatttgttgttggcgtttggggtgaggtttgatgatcgcgtcacgggtaagcttgaggcttttgctagtagggctaagattgttcatattgatattgactctgctgagattgggaagaataagactcctcatgtgtctgtgtgtggtgatgtcaagctggctttgcaagggatgaataaggttcttgagaaccgagctgaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaagccaaggcagtggctatcatcaggaggccttggagctatgggttttggacttcctgctgccattggagcgtctgttgctaaccctgatgcaatagttgtggatattgacggagatggaagctttataatgaatgtgcaagagctggccacaatccgtgtagagcaacttccagtgaagatactcttattaaacaaccagcatcttggcatggttatgcaatgggaagatcggttctacaaggctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactga

SEQ ID NO: 14 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVEQLPVKILLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

(19) United States
(12) Patent Application Publication
(10) Pub. No.: US 2016/0222403 A1
(43) Pub. Date: Aug. 4, 2016

(54) HERBICIDE-RESISTANT SUNFLOWER PLANTS, POLYNUCLEOTIDES ENCODING HERBICIDE-RESISTANT ACETOHYDROXYACID SYNTHASE LARGE SUBUNIT PROTEINS, AND METHODS OF USE

(71) Applicants: BASF Aktiengesellschaft, Ludwigshafen (DE); Nidera S.A., Buenos Aires (AR)

(72) Inventors: Carlos Alberto SALA, Santa Fe (AR); Mariano Bulos, Santa Fe (AR); Sherry R. Whitt, Raleigh, NC (US); Robert Ascenzi, Cary, NC (US); Adriana Mariel Echarte, Santa Fe (AR)

(73) Assignees: BASF Aktiengesellschaft, Ludwigshafen (DE); Nidera S.A., Buenos Aires (AR)

(21) Appl. No.: 15/054,883

(22) Filed: Feb. 26, 2016

Related U.S. Application Data
(62) Division of application No. 11/993,725, filed on Feb. 21, 2011.
(60) Provisional application No. 60/695,952, filed on Jul. 1, 2005.

(51) Int. Cl. C12N 15/82; A01N 43/50; C12Q 1/68

(52) U.S. Cl. CPC C12N 15/8274; C12N 15/8278; C12Q 1/6895; A01N 43/50; C12Q 2600/13; C12Q 2600/158

(57) ABSTRACT

Herbicide-resistant sunflower plants, isolated polynucleotides that encode herbicide-resistant and wild-type acetohydroxyacid synthase large subunit (AHASL) polypeptides, and the amino acid sequences of these polypeptides, are described. Expression cassettes and transformation vectors comprising the polynucleotides of the invention, as well as plants and host cells transformed with the polynucleotides, are described. Methods of using the polynucleotides to enhance the resistance of plants to herbicides, and methods for controlling weeds in the vicinity of herbicide-resistant plants are also described.

### FIELD OF THE INVENTION

This invention relates to the field of agricultural biotechnology, particularly to herbicide-resistant sunflower plants and novel polynucleotide sequences that encode wild-type and herbicide-resistant sunflower acetohydroxyacid synthase large subunit proteins.

### BACKGROUND OF THE INVENTION

Acetohydroxyacid synthase (AHAS; EC 4.1.3.18, also known as acetolactate synthase or ALS), is the first enzyme that catalyzes the biochemical synthesis of the branched chain amino acids valine, leucine and isoleucine (Singh (1999) “Biosynthesis of valine, leucine and isoleucine,” in Plant Amino Acid, Singh, B. K., ed., Marcel Dekker Inc. New York, N.Y., pp. 227-247). AHAS is the site of action of five structurally diverse herbicide families including the sulfonylureas (Tan et al. (2005) Pest Manag. Sci. 61:246-57; Mallory-Smith and Retzinger (2003) Weed Technology 17:620-626; LaRossa and Falco (1984) Trends Biotechnol. 2:158-161), the imidazolinones (Shaner et al. (1984) Plant Physiol. 76:545-546), the triazolopyrimidines (Subramanian and Gerwick (1989) “Inhibition of acetolactate synthase by triazolopyrimidines,” in Biocatalysis in Agricultural Biotechnology, Whitaker, J. R. and Sonnet, P. E. eds., ACS Symposium Series, American Chemical Society, Washington, D.C., pp. 277-288), the pyrimidinyloxybenzoates (Subramanian et al (1990) Plant Physiol. 94: 239-244) and the sulfonylarnino-carbonyltriazolinones (Tan et al. (2005) Pest Manag. Sci. 61:246-57; Mallory-Smith and Retzinger (2003) Weed Technology 17:620-626). Imidazolinone and sulfonylurea herbicides are widely used in modern agriculture due to their effectiveness at very low application rates and relative non-toxicity in animals. By inhibiting AHAS activity, these families of herbicides prevent further growth and development of susceptible plants including many weed species. Several examples of commercially available imidazolinone herbicides are PURSUIT® (imazethapyr), SCEPTER® (imazaquin) and ARSENAL® (imazapyr). Examples of sulfonylurea herbicides are chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfiuon, fluzasulfuron, imazosulfuron, pyrazosulfuron ethyl and halosulfuron.

Due to their high effectiveness and low-toxicity, imidazolinone herbicides are favored for application by spraying over the top of a wide area of vegetation. The ability to spray a herbicide over the top of a wide range of vegetation decreases the costs associated with plant establishment and maintenance, and decreases the need for site preparation prior to use of such chemicals. Spraying over the top of a desired tolerant species also results in the ability to achieve maximum yield potential of the desired species due to the absence of competitive species. However, the ability to use such spray-over techniques is dependent upon the presence of imidazolinone-resistant species of the desired vegetation in the spray over area.

Among the major agricultural crops, some leguminous species such as soybean are naturally resistant to imidazolinone herbicides due to their ability to rapidly metabolize the herbicide compounds (Shaner and Robinson (1985) Weed Sci. 33:469-471). Other crops such as corn (Newhouse et al. (1992) Plant Physiol. 100:882-886) and rice (Barrett et al. (1989) Crop Safeners for Herbicides, Academic Press, New York, pp. 195-220) are somewhat susceptible to imidazolinone herbicides. The differential sensitivity to the imidazolinone herbicides is dependent on the chemical nature of the particular herbicide and differential metabolism of the compound from a toxic to a non-toxic form in each plant (Shaner et al. (1984) Plant Physiol. 76:545-546; Brown et al., (1987) Pestic. Biochem. Physiol. 27:24-29). Other plant physiological differences such as absorption and translocation also play an important role in sensitivity (Shaner and Robinson (1985) Weed Sci. 33:469-471).

Plants resistant to imidazolinones, sulfonylureas, triazolopyrimidines, and pyrimidinyloxybenzoates have been successfully produced using seed, microspore, pollen, and callus mutagenesis in Zea mays, Arabidopsis thaliana, Brassica napes (i. e., canola) Glycine max, Nicotiana tabacum, sugarbeet (Beta vulgaris) and Oryza sativa (Sebastian et al. (1989) Crop Sci. 29:1403-1408; Swanson et al., 1989 Theor. Appl. Genet. 78:525-530; Newhouse et al. (1991) Theor. Appl. Genet. 83:65-70; Sathasivan et al. (1991) Plant Physiol. 97:1044-1050; Mourand et al. (1993) J. Heredity 84:91-96; Wright and Penner (1998) Theor. Appl. Genet. 96:612-620; U.S. Pat. No. 5,545,822). In all cases, a single, partially dominant nuclear gene conferred resistance. Four imidazolinone resistant wheat plants were also previously isolated following seed mutagenesis of Triticum aestivum L. cv. Fidel (Newhouse et al. (1992) Plant Physiol. 100:882-886). Inheritance studies confirmed that a single, partially dominant gene conferred resistance. Based on allelic studies, the authors concluded that the mutations in the four identified lines were located at the same locus. One of the Fidel cultivar resistance genes was designated FS-4 (Newhouse et al. (1992) Plant Physiol. 100:882-886).

Naturally occurring plant populations that were discovered to be resistant to imidazolinone and/or sulfonylurea herbicides have also been used to develop herbicide-resistant sunflower breeding lines. Recently, two sunflower lines that are resistant to a sulfonylurea herbicide were developed using germplasm originating from a wild population of common sunflower (Helianthus annuus) as the source of the herbicide-resistance trait (Miller and Al-Khatib (2004) Crop Sci. 44:1037-1038). Previously, White et al. ((2002) Weed Sci. 50:432-437) had reported that individuals from a wild population of common sunflower from South Dakota, U.S.A. were cross-resistant to an imidazolinone and a sulfonylurea herbicide. Analysis of a portion of the coding region of the acetohydroxyacid synthase large subunit (AHASL) genes of individuals from this population revealed a point mutation that results in an Ala-to-Val amino acid substitution in the sunflower AHASL protein that corresponds to Ala₂₀₅ in the wild-type Arabidopsis thaliana AHASL protein (White et al. (2003) Weed Sci. 51:845-853). Earlier, Al-Khatib and Miller ((2000) Crop Sci. 40:869) reported the production of four imidazolinone-resistant sunflower breeding lines.

Computer-based modeling of the three dimensional conformation of the AHAS-inhibitor complex predicts several amino acids in the proposed inhibitor binding pocket as sites where induced mutations would likely confer selective resistance to imidazolinones (Ott et al. (1996) J. Mol. Biol. 263:359-368). Tobacco plants produced with some of these rationally designed mutations in the proposed binding sites of the AHAS enzyme have in fact exhibited specific resistance to a single class of herbicides (Ott et al. (1996) J. Mol. Biol. 263:359-368).

Plant resistance to imidazolinone herbicides has also been reported in a number of patents. U.S. Pat. Nos. 4,761,373, 5,331,107, 5,304,732, 6,211,438, 6,211,439 and 6,222,100 generally describe the use of an altered AHAS gene to elicit herbicide resistance in plants, and specifically discloses certain imidazolinone resistant corn lines. U.S. Pat. No. 5,013,659 discloses plants exhibiting herbicide resistance due to mutations in at least one amino acid in one or more conserved regions. The mutations described therein encode either cross-resistance for imidazolinones and sulfonylureas or sulfonylurea-specific resistance, but imidazolinone-specific resistance is not described. U.S. Pat. No. 5,731,180 and U.S. Pat. No. 5,767,361 discuss an isolated gene having a single amino acid substitution in a wild-type monocot AHAS amino acid sequence that results in imidazolinone-specific resistance. In addition, rice plants that are resistant to herbicides that interfere with AHAS have been developed by mutation breeding and also by the selection of herbicide-resistant plants from a pool of rice plants produced by anther culture. See, U.S. Pat. Nos. 5,545,822, 5,736,629, 5,773,703, 5,773,704, 5,952,553 and 6,274,796.

In plants, as in all other organisms examined, the AHAS enzyme is comprised of two subunits: a large subunit (catalytic role) and a small subunit (regulatory role) (Duggleby and Pang (2000) J. Biochem. Mol. Biol. 33:1-36). The AHAS large subunit (also referred to herein as AHASL) may be encoded by a single gene as in the case of Arabidopsis, and sugar beet or by multiple gene family members as in maize, canola, and cotton. Specific, single-nucleotide substitutions in the large subunit confer upon the enzyme a degree of insensitivity to one or more classes of herbicides (Chang and Duggleby (1998) Biochem J. 333:765-777).

For example, bread wheat, Triticum aestivum L., contains three homoeologous acetohydroxyacid synthase large subunit genes. Each of the genes exhibit significant expression based on herbicide response and biochemical data from mutants in each of the three genes (Ascenzi et al. (2003) International Society of Plant Molecular Biologists Congress, Barcelona, Spain, Ref. No. S10-17). The coding sequences of all three genes share extensive homology at the nucleotide level (WO 03/014357). Through sequencing the AHASL genes from several varieties of Triticum aestivum, the molecular basis of herbicide tolerance in most IMI-tolerant (imidazolinone-tolerant) lines was found to be the mutation S653(At)N, indicating a serine to asparagine substitution at a position equivalent to the serine at amino acid 653 in Arabidopsis thaliana (WO 03/01436; WO 03/014357). This mutation is due to a single nucleotide polymorphism (SNP) in the DNA sequence encoding the AHASL protein.

Multiple AHASL genes are also know to occur in dicotyledonous plants species. Recently, Kolkman et al. ((2004) Theor. Appl. Genet. 109: 1147-1159) reported the identification, cloning, and sequencing for three AHASL genes (AHASL1, AHASL2, and AHASL3) from herbicide-resistant and wild type genotypes of sunflower (Helianthus annuus L.). Kolkman et al. reported that the herbicide-resistance was due either to the Pro197Leu (using the Arabidopsis AHASL amino acid position nomenclature) substitution or the Ala205Val substitution in the AHASL1 protein and that each of these substitutions provided resistance to both imidazolinone and sulfonylurea herbicides.

Given their high effectiveness and low-toxicity, imidazolinone herbicides are favored for agricultural use. However, the ability to use imidazolinone herbicides in a particular crop production system depends upon the availability of imidazolinone-resistant varieties of the crop plant of interest. To produce such imidazolinone-resistant varieties, plant breeders need to develop breeding lines with the imidazolinone-resistance trait. Thus, additional imidazolinone-resistant breeding lines and varieties of crop plants, as well as methods and compositions for the production and use of imidazolinone-resistant breeding lines and varieties, are needed.

### SUMMARY OF THE INVENTION

The present invention provides sunflower plants having increased resistance to herbicides when compared to a wild-type sunflower plant. In particular, the sunflower plants of the invention have increased resistance to imidazolinone herbicides, when compared to a wild-type sunflower plant. The herbicide-resistant sunflower plants of the invention comprise at least one copy of a gene or polynucleotide that encodes a herbicide-resistant acetohydroxyacid synthase large subunit (AHASL). Such a herbicide-resistant AHASL protein comprises a threonine at amino acid position 107 or equivalent position. A herbicide-resistant sunflower plant of the invention can contain one, two, three, four, five, six, or more copies of a gene or polynucleotide encoding a herbicide-resistant AHASL protein of the invention. The sunflower plants of the invention also include seeds and progeny plants that comprise at least one copy of a gene or polynucleotide encoding a herbicide-resistant AHASL protein of the invention.

In one embodiment, the present invention provides herbicide-resistant sunflower plants of a sunflower line that is designated herein as S4897 and progeny and derivatives thereof that comprise the herbicide resistance characteristics of S4897. A genetic line designated herein as GM40 was derived from, and possesses the herbicide resistance characteristics of S4897. A sample of seeds of the GM40 line has been deposited with the American Type Culture Collection (ATCC) as ATCC Patent Deposit No. PTA-6716. Thus, a sunflower plant of the invention that comprises the herbicide resistance characteristics of GM40 or a sunflower plant having ATCC Patent Deposit No. PTA-6716 also comprises the herbicide resistance characteristics of S4897. S4897 sunflower plants, GM40 sunflower plants, and sunflower plants having ATCC Patent Deposit No. PTA-6716 and progeny and derivatives thereof comprising the herbicide resistance characteristics of S4897, GM40, or a sunflower plant having ATCC Patent Deposit No. PTA-6716, comprise in their genomes an AHASL1 gene that comprises the nucleotide sequence set forth in SEQ ID NO: 1 and that encodes the AHASL1 protein comprising, the amino acid sequence set forth in SEQ ID NO: 2. When compared to the amino acid sequence of the AHASL1 protein (SEQ ID NO: 4) that is encoded by an AHASL1 gene (SEQ ID NO: 3) from a wild-type sunflower plant, the amino acid sequence set forth in SEQ ID NO: 2 has a single amino acid difference from the wild-type amino acid sequence. In the amino acid sequence set forth in SEQ ID NO: 2, there is threonine at amino acid position 7. This position corresponds to position 107 in the full-length sunflower AHASL1 protein encoded by the nucleotide sequence set forth in SEQ ID NO: 12 (Accession No. AY541451). In the wild-type AHASL1 amino acid sequence of the invention (SEQ ID NO: 4), this equivalent amino acid position has an alanine. Unless otherwise indicated, the amino acid positions referred to herein for sunflower AHASL proteins correspond to the amino acid positions of the full-length amino acid sequence set forth in SEQ ID NO: 12.

In another embodiment, the present invention provides herbicide-resistant sunflower plants that are sunflower line that is designated herein as GM1606. A sample of seeds of genetic material of GM1606 has been deposited with the ATCC as ATCC Patent Deposit No. PTA-7606. Thus, the present invention provides herbicide-resistant sunflower plants that having ATCC Patent Deposit No. PTA-7606 and progeny and derivatives thereof that comprise the herbicide resistance characteristics of the sunflower plants having ATCC Patent Deposit No. PTA-7606. Like the S4897 and GM40 sunflower plants, GM1606 and progeny and derivatives thereof comprising the herbicide resistance characteristics of GM1606 comprise in their genomes an AHASL1 gene encodes an AHASL1 protein comprising a threonine at position 107 in the full-length sunflower AHASL1 protein. Similarly, sunflower plants having ATCC Patent Deposit No. PTA-7606, and progeny and deriviatives thereof comprising the herbicide resistance characteristics of the sunflower plants having ATCC Patent Deposit No. PTA-7606 comprise in their genomes an AHASL1 gene encodes an AHASL1 protein comprising a threonine at position 107 in the full-length sunflower AHASL1 protein.

The present invention further provides isolated polynucleotides and isolated polypeptides for sunflower (Helianthus annuus) AHASL proteins. The polynucleotides of the invention encompass nucleotide sequences that encode herbicide-resistant and wild-type AHASL proteins, including, but not limited to, the proteins encoded by the sunflower AHASL1, AHASL2, and AHASL3 genes. The herbicide-resistant sunflower AHASL proteins of the invention are imidazolinone-resistant AHASL proteins that comprise an amino acid other than alanine at position 107 of a full-length sunflower AHASL1 protein or equivalent position. Preferably, the amino acid at position 107 or equivalent position is a threonine. The polynucleotides of the invention encompass the nucleotide sequences set forth in SEQ ID NOS: 1 and 3, nucleotide sequences encoding the amino acid sequences set forth in SEQ ID NOS: 2 and 4, and fragments and variants of said nucleotide sequences that encode proteins comprising AHAS activity.

The present invention provides expression cassettes for expressing the polynucleotides of the invention in plants, plant cells, and other, non-human host cells. The expression cassettes comprise a promoter expressible in the plant, plant cell, or other host cells of interest operably linked to a polynucleotide of the invention that encodes either a wild-type or herbicide-resistant AHASL protein. If necessary for targeting expression to the chloroplast, the expression cassette can also comprise an operably linked chloroplast-targeting sequence that encodes of a chloroplast transit peptide to direct an expressed AHASL protein to the chloroplast. The expression cassettes of the invention find use in a method for enhancing the herbicide tolerance of a plant and a host cell. The method involves transforming the plant or host cell with an expression cassette of the invention, wherein the expression cassette comprises a promoter that is expressible in the plant or host cell of interest and the promoter is operably linked to a polynucleotide of the invention that encodes an herbicide-resistant AHASL protein of the invention. The method further comprises regenerating a transformed plant from the transformed plant cell.

The present invention provides a method for increasing AHAS activity in a plant comprising transforming a plant cell with a polynucleotide construct comprising a nucleotide sequence operably linked to a promoter that drives expression in a plant cell and regenerating a transformed plant from the transformed plant cell. The nucleotide sequence is selected from those nucleotide sequences that encode the herbicide-resistant or wild-type AHASL proteins of the invention, particularly the nucleotide sequences set forth in SEQ ID NOS: 1 and 3, and nucleotide sequences encoding the amino acid sequences set forth in SEQ ID NOS: 2, and 4, and fragments and variants thereof. A plant produced by this method comprises increased AHAS activity or increased herbicide-resistant AHAS activity, when compared to an untransformed plant.

The present invention provides a method for producing a herbicide-resistant plant comprising transforming a plant cell with a polynucleotide construct comprising a nucleotide sequence operably linked to a promoter that drives expression in a plant cell and regenerating a transformed plant from said transformed plant cell. The nucleotide sequence is selected from those nucleotide sequences that encode the herbicide-resistant AHASL proteins of the invention, particularly the nucleotide sequence set forth in SEQ ID NO: 1, the nucleotide sequence encoding the amino acid sequence set forth in SEQ ID NO: 2, and fragments and variants thereof. A herbicide-resistant plant produced by this method comprises enhanced resistance to at least one herbicide, particularly an imidazolinone herbicide, when compared to an untransformed plant.

The present invention provides a method for enhancing herbicide-tolerance in a herbicide-tolerant plant. The method finds use in enhancing the resistance of a plant that already is resistant to a level of a herbicide that would kill or significantly injure a wild-type plant. Such a herbicide-tolerant plant can be a herbicide-tolerant plant that has been genetically engineered for herbicide-tolerance or a herbicide-tolerant plant that was developed by means that do not involve recombinant DNA such as, for example, the S4897, GM40, and GM1606 sunflower plants of the present invention. The method comprises transforming a herbicide-tolerant plant with a polynucleotide construct comprising a nucleotide sequence operably linked to a promoter that drives expression in a plant cell and regenerating a transformed plant from the transformed plant cell. The nucleotide sequence is selected from those nucleotide sequences that encode the herbicide-resistant AHASL proteins of the invention, particularly a nucleotide sequence comprising the nucleotide sequence set forth in SEQ ID NO: 1, nucleotide sequences encoding an amino acid sequence comprising the amino acid sequence set forth in SEQ ID NO: 2, and fragments and variants thereof.

The present invention provides transformation vectors comprising a selectable marker gene of the invention. The selectable marker gene comprises a promoter that drives expression in a host cell operably linked to a polynucleotide comprising a nucleotide sequence that encodes an herbicide-resistant AHASL protein of the invention. The transformation vector can additionally comprise a gene of interest to be expressed in the host cell and can also, if desired, include a chloroplast-targeting sequence that is operably linked to the polynucleotide of the invention.

The present invention further provides methods for using the transformation vectors of the invention to select for cells transformed with the gene of interest. Such methods involve the transformation of a host cell with the transformation vector, exposing the cell to a level of an imidazolinone herbicide that would kill or inhibit the growth of a non-transformed host cell, and identifying the transformed host cell by its ability to grow in the presence of the herbicide. In one embodiment of the invention, the host cell is a plant cell and the selectable marker gene comprises a promoter that drives expression in a plant cell.

The present invention provides a method for controlling weeds in the vicinity of the herbicide-resistant plants of the invention, including the herbicide-resistant sunflower plants described above and plants transformed with the herbicide-resistant AHASL polynucleotides of the invention. Such transformed plants comprise in their genomes at least one expression cassette comprising a promoter that drives gene expression in a plant cell, wherein the promoter is operably linked to an AHASL polynucleotide of the invention. The method comprises applying an effective amount of an herbicide to the weeds and to the herbicide-resistant plant, wherein the herbicide-resistant plant has increased resistance to at least one herbicide, particularly an imidazolinone herbicide, when compared to a wild-type or untransformed plant.

The plants of the present invention can be transgenic or non-transgenic. An example of a non-transgenic sunflower plant having increased resistance to imidazolinone and/or sulfonylurea herbicides includes S4897, GM40, or GM1606 sunflower plants and sunflower plants having ATCC Patent Deposit No. PTA-6716 or PTA-7606; or mutant, recombinant, or a genetically engineered derivative of and S4897, GM40, or GM1606, the sunflower plant having ATCC Patent Deposit No. PTA-6716 or PTA-7606, or two of more of S4897, GM40, GM1606, the sunflower plant having ATCC Patent Deposit No. PTA-6716, and the sunflower plant having ATCC Patent Deposit No. PTA-7606; or of any progeny of S4897, GM40, or GM1606, the sunflower plant having ATCC Patent Deposit No. PTA-6716 or PTA-7606, or two of more of S4897, GM40, GM1606, the sunflower plant having ATCC Patent Deposit No. PTA-6716, and the sunflower plant having ATCC Patent Deposit No. PTA-7606; or a plant that is a progeny of any of these plants; or a plant that comprises the herbicide resistance characteristics of S4897, GM40, GM1606, the sunflower plant having ATCC Patent Deposit No. PTA-6716, and/or the sunflower plant having ATCC Patent Deposit No. PTA-7606.

The present invention also provides plants, plant organs, plant tissues, plant cells, seeds, and non-human host cells that are transformed with the at least one polynucleotide, expression cassette, or transformation vector of the invention. Such transformed plants, plant organs, plant tissues, plant cells, seeds, and non-human host cells have enhanced tolerance or resistance to at least one herbicide, at levels of the herbicide that kill or inhibit the growth of an untransformed plant, plant tissue, plant cell, or non-human host cell, respectively. Preferably, the transformed plants, plant tissues, plant cells, and seeds of the invention are Arabidopsis thaliana, sunflower, and other crop plants.

The present invention further provides isolated polypeptides comprising imidazolinone-resistant and wild-type sunflower AHASL proteins. The isolated polypeptides comprise the amino acid sequences set forth in SEQ ID NOS: 2 and 4, the amino acid sequences encoded by nucleotide sequences set forth in SEQ ID NOS: 1 and 3, and fragments and variants of said amino acid sequences that encode proteins comprising AHAS activity.

### BRIEF DESCRIPTION THE DRAWINGS

FIG. 1 is a nucleotide sequence alignment of the nucleotide sequences of the herbicide-resistant sunflower AHASL1 gene (SEQ ID NO: 1), the wild-type sunflower AHASL1 gene (SEQ ID NO: 3), GenBank Accession No. U16280 (SEQ ID NO: 5), GenBank Accession No. AY541451 (SEQ ID NO: 11) and GenBank Accession No. AY124092 (SEQ ID NO: 13). The site of the mutation in (SEQ ID NO: 1) is indicated by an asterisk. The mutation is a G-to-A transition at nucleotide position 21 of SEQ ID NO: 1.

FIG. 2 is an amino acid sequence alignment of the herbicide-resistant sunflower AHASL1 protein (SEQ ID NO: 2), the wild-type sunflower AHASL1 protein (SEQ ID NO: 4), GenBank Accession No. U16280 (SEQ ID NO: 6), GenBank Accession No. AY541451 (SEQ ID NO: 12), and GenBank Accession No. AY124092 (SEQ ID NO: 14). The asterisk indicates the site of the single amino acid substitution (Ala-to-Thr) found in the herbicide-resistant sunflower AHASL1 protein. This site of the substitution corresponds to amino acid position 7 in the partial-length AHASL1 amino acid sequence set forth in SEQ ID NO: 2. The equivalent position of this substitution in the full-length sunflower AHASL1 amino acid sequence set forth in SEQ ID NO: 12 is 107.

FIG. 3 is a photographic illustration demonstrating the increased herbicide tolerance of S4897 sunflower plants (right side) plants as compared to the herbicide-tolerant IMISUN-1 sunflower plants (Al-Khatib and Miller (2000) Crop Sci. 40:869-870) in a greenhouse study. The S4897 and IMISUN-1 sunflower plants were spray-treated with imazamox at rate of 200 g ai/ha. Control, wild-type sunflower plants did not survive following the spray treatment with imazamox at a rate of either 100 or 200 g ai/ha (not shown). The photograph was taken a few days after the plants were spray treated.

FIG. 4 is graphical illustration of herbicide injury in a greenhouse test following the spray treatment of IMISUN-1, wild-type, and S4897 sunflower plants with imazamox at either 100 (light columns) or 200 g ai/ha (dark columns). This figure demonstrates that the S4897 herbicide-resistance sunflower plants have a significantly greater resistance or tolerance to two rates of imazamox when compared either the herbicide-resistant IMISUN-1 plants and wild-type plants. Herbicide injury was evaluated 17 days after the imazamox application. The IMISUN-1 plants are known to possess an alanine-to-valine substitution at amino acid 190 (Kolkman et al. (2004) Theor. Appl. Genet. 109: 1147-1159).

FIG. 5 is a photographic illustration demonstrating the increased herbicide tolerance of S4897 sunflower plants (right side) as compared to the herbicide-tolerant IMISUN-1 sunflower plants in the greenhouse trial described in Example 4.

FIG. 6 is a photographic illustration demonstrating the increased herbicide tolerance of S4897 sunflower plants as compared to Clearfield® Variety A sunflower plants in the greenhouse trial described in Example 5.

FIG. 7 is graphical illustration of a comparison of the inhibition of AHAS by Raptor for a non-Clearfield variety, a Clearfield variety and S4897 sunflower plants as described in Example 5.

FIG. 8 is graphical illustration of a comparison of the inhibition of AHAS by Glean for a non-Clearfield variety, a Clearfield variety and S4897 sunflower plants as described in Example 5.

FIG. 9 is a graphical illustration of the effect of foliar application of imazapir on plant height 14 days after treatment for two mutants of sunflower. Mean height (% of untreated plots) are represented by squares and error bars represent the standard deviation of the means.

FIG. 10 is a graphical illustration of the effect of foliar application of imazapir on Phytotoxicity Index (PI) 14 days after treatment for two mutants of sunflower. Mean PI are represented by squares and error bars represent the standard deviation of the means.

FIG. 11 is a graphical illustration of the effect of foliar application of imazapir on biomass accumulation 14 days after treatment for two mutants of sunflower. Mean dry biomass (% of untreated plots) are represented by squares and error bars represent the standard deviation of the means.

FIG. 12 is a graphical illustration of the effect of foliar application of imazapir on root biomass 14 days after treatment for two mutants of sunflower. Mean root dry mass (% of untreated plots) are represented by squares and error bars represent the standard deviation of the means.

### SEQUENCE LISTING

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleic acid sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO: 1 sets forth the partial-length nucleotide sequence encoding a herbicide-resistant AHASL1 protein from the sunflower line S4897.

SEQ ID NO: 2 sets forth the partial-length amino acid sequence of the herbicide-resistant AHASL1 protein encoded by the nucleotide sequence set forth in SEQ ID NO: 1.

SEQ ID NO: 3 sets forth the partial-length nucleotide sequence encoding the wild-type AHASL1 protein from sunflower line BTK47.

SEQ ID NO: 4 sets forth the partial-length amino acid sequence of the wild-type AHASL1 protein encoded by the nucleotide sequence set forth SEQ ID NO: 3.

SEQ ID NO: 5 is the nucleotide sequence of GenBank Accession No. U16280.

SEQ ID NO: 6 is the amino acid sequence encoded by the nucleotide sequence of GenBank Accession No. U16280.

SEQ ID NO: 7 sets forth the nucleotide sequence of the HA1U409 primer that is described in Example 2.

SEQ ID NO: 8 sets forth the nucleotide sequence of the HA1L1379 primer that is described in Example 2.

SEQ ID NO: 9 sets forth the nucleotide sequence of the HA1U1313 primer that is described in Example 2.

SEQ ID NO: 10 sets forth the nucleotide sequence of the HA1L2131 primer that is described in Example 2.

SEQ ID NO: 11 is the nucleotide sequence of GenBank Accession No. AY541451.

SEQ ID NO: 12 is the amino acid sequence encoded by the nucleotide sequence of GenBank Accession No. AY541451.

SEQ ID NO: 13 is the nucleotide sequence of GenBank Accession No. AY124092.

SEQ ID NO: 14 is the amino acid sequence encoded by the nucleotide sequence of GenBank Accession No. AY124092.

### DETAILED DESCRIPTION OF THE INVENTION

The present invention relates to sunflower plants having increased resistance to herbicides when compared to a wild-type sunflower plant. Herbicide-resistant sunflower plants were produced as described hereinbelow by exposing wild-type (with respect to herbicide resistance) sunflower plants to a mutagen, allowing the plants to mature and reproduce, and selecting progeny plants that displayed enhanced resistance to an imidazolinone herbicide, relative to the resistance of a wild-type sunflower plant. The invention provides the herbicide-resistant sunflower lines that are referred to herein as S4897, GM40, and GM1606.

From the S4897 herbicide-resistant sunflower plants and BTK47 wild-type sunflower plants, the coding region of an acetohydroxyacid synthase large subunit gene (designated as AHASL1) was isolated by polymerase chain reaction (PCR) amplification and sequenced. By comparing the polynucleotide sequences of the herbicide-resistant and wild-type sunflower plants, it was discovered that the coding region of the AHASL1 polynucleotide sequence from the herbicide-resistant sunflower plant differed from the AHASL1 polynucleotide sequence of the wild type plant by a single nucleotide, a G-to-A transition at nucleotide 21 (SEQ ID NO: 1). This G-to-A transition in the AHASL1 polynucleotide sequence results in an alanine-to-threonine substitution at amino acid 7 (SEQ ID NO: 2) in a conserved region of the predicted amino acid sequence of the herbicide-resistant sunflower AHASL1 protein (SEQ ID NO: 2), relative to the equivalent amino acid position of the wild-type AHASL1 protein from sunflower line BTK47 (i.e., amino acid 7 of SEQ ID NO: 4).

Because the nucleotide sequence set forth in SEQ ID NO: 1 does not correspond to a full-length coding region of an AHASL protein, the amino acid sequence encoded thereby that is set forth in SEQ ID NO: 2 is also less than full-length. To facilitate comparison with other sunflower AHASL amino acid sequences, the amino acid positions of sunflower AHASL proteins referred to herein, unless otherwise indicated or apparent for the context in which such positions appear, correspond to the amino acid positions of the full-length amino acid sequence of the sunflower AHASL1 protein encoded by the nucleotide sequence having GenBank Accession No. AY541451 (SEQ ID NO: 12). Accordingly, the alanine-to-threonine substitution at amino acid position 7 of SEQ ID NO: 2 corresponds to amino acid position 107 in the amino acid sequence of SEQ ID NO: 12.

Thus, the present invention discloses an amino acid substitution that can be used to produce herbicide-resistant sunflower AHASL proteins, the polynucleotides encoding such proteins, and herbicide-resistant plants, plant tissues, plant cells, and seeds. Because the alanine that is found at amino acid position 107 in wild-type, full-length sunflower AHASL1 proteins or equivalent position is within a region of amino acids that is conserved across plant species, it is expected that the substitution of another amino acid, preferably threonine, for this same conserved alanine in other AHASL proteins from sunflower (e.g., AHASL2 and AHASL3) will also confer herbicide tolerance. Thus, a polynucleotide encoding a sunflower AHASL protein can be mutated by any method known in the art such as, for example, site-directed mutagenesis to produce a sunflower polynucleotide that encodes an AHASL protein with a threonine at position 107 or equivalent position. Polynucleotide sequences and the amino acid sequences encoded thereby corresponding to the sunflower AHASL1, AHASL2, and AHASL3 genes are set forth in GenBank Accession Nos. AY541451 (SEQ ID NOS: 11 and 12), AY541452, AY541453, AY541454, AY541455, AY541456, AY541457, and AY541458; all of which are herein incorporated by reference. Accordingly, such polynucleotides and the herbicide-resistant AHASL proteins encoded thereby find use in the production of herbicide-resistant plants, plant cells, plant tissues, and seeds by the methods disclosed herein.

The present invention additionally encompasses isolated sunflower AHASL2 and AHASL3 polynucleotides that encode herbicide-resistant AHASL2 and AHASL3 proteins, respectively. Such herbicide-resistant AHASL2 and AHASL3 proteins each comprise an amino acid other than alanine at position 107 or equivalent position. Preferably in such herbicide-resistant AHASL2 and AHASL3 proteins, the amino acid at position 107 or equivalent position is threonine.

The invention further relates to isolated polynucleotide molecules comprising nucleotide sequences that encode acetohydroxyacid synthase large subunit (AHASL) proteins and to such AHASL proteins. The invention discloses the isolation and nucleotide sequence of a polynucleotide encoding a herbicide-resistant sunflower AHASL1 protein from an herbicide-resistant sunflower plant that was produced by chemical mutagenesis of wild-type sunflower plants. The herbicide-resistant sunflower AHASL1 proteins of the invention possess a alanine-to-threonine substitution at position 107 or equivalent position in their respective amino acid sequences, when compared to the corresponding wild-type amino acid sequence. The invention further discloses the isolation and nucleotide sequence of a polynucleotide molecule encoding a wild-type sunflower AHASL1 protein.

The present invention provides isolated polynucleotide molecules that encode AHASL proteins from sunflower (Helianthus annuus L.). Specifically, the invention provides isolated polynucleotide molecules comprising: the nucleotide sequences set forth in SEQ ID NOS: 1 and 3, nucleotide sequences encoding AHASL proteins comprising the amino acid sequences set forth in SEQ ID NOS: 2 and 4, and fragments and variants of such nucleotide sequences that encode functional AHASL proteins.

The isolated herbicide-resistant AHASL polynucleotide molecules of the invention comprise nucleotide sequences that encode herbicide-resistant AHASL proteins. Such polynucleotide molecules can be used in polynucleotide constructs for the transformation of plants, particularly crop plants, to enhance the resistance of the plants to herbicides, particularly herbicides that are known to inhibit AHAS activity, more particularly imidazolinone herbicides. Such polynucleotide constructs can be used in expression cassettes, expression vectors, transformation vectors, plasmids and the like. The transgenic plants obtained following transformation with such polynucleotide constructs show increased resistance to AHAS-inhibiting herbicides such as, for example, imidazolinone and sulfonylurea herbicides.

Compositions of the invention include nucleotide sequences that encode AHASL proteins. In particular, the present invention provides for isolated polynucleotide molecules comprising nucleotide sequences encoding the amino acid sequences shown in SEQ ID NOS: 2 and 4, and fragments and variants thereof that encode polypeptides comprising AHAS activity. Further provided are polypeptides having an amino acid sequence encoded by a polynucleotide molecule described herein, for example those set forth in SEQ ID NOS: 1 and 3, and fragments and variants thereof that encode polypeptides comprising AHAS activity.

The invention encompasses isolated or substantially purified nucleic acid or protein compositions. An “isolated” or “purified” polynucleotide molecule or protein, or biologically active portion thereof, is substantially or essentially free from components that normally accompany or interact with the polynucleotide molecule or protein as found in its naturally occurring environment. Thus, an isolated or purified polynucleotide molecule or protein is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated polynucleotide molecule can contain less than about 5 kb, 4 kb, 3 kb, 2 kb, 1 kb, 0.5 kb, or 0.1 kb of nucleotide sequences that naturally flank the polynucleotide molecule in genomic DNA of the cell from which the nucleic acid is derived. A protein that is substantially free of cellular material includes preparations of protein having less than about 30%, 20%, 10%, 5%, or 1% (by thy weight) of contaminating protein. When the protein of the invention or biologically active portion thereof is recombinantly produced, preferably culture medium represents less than about 30%, 20%, 10%, 5%, or 1% (by dry weight) of chemical precursors or non-protein-of-interest chemicals.

The present invention provides isolated polypeptides comprising AHASL proteins. The isolated polypeptides comprise an amino acid sequence selected from the group consisting of the amino acid sequences set forth in SEQ ID NOS: 2 and 4, the amino acid sequences encoded by nucleotide sequences set forth in SEQ ID NOS: 1 and 3, and functional fragments and variants of said amino acid sequences that encode an AHASL polypeptide comprising AHAS activity. By “functional fragments and variants” is intended fragments and variants of the exemplified polypeptides that comprise AHAS activity.

In certain embodiments of the invention, the methods involve the use of herbicide-tolerant or herbicide-resistant plants. By an “herbicide-tolerant” or “herbicide-resistant” plant, it is intended that a plant that is tolerant or resistant to at least one herbicide at a level that would normally kill, or inhibit the growth of, a normal or wild-type plant. In one embodiment of the invention, the herbicide-tolerant plants of the invention comprise a herbicide-tolerant or herbicide-resistant AHASL protein. By “herbicide-tolerant AHASL protein” or “herbicide-resistant AHASL protein”, it is intended that such an AHASL protein displays higher AHAS activity, relative to the AHAS activity of a wild-type AHASL protein, when in the presence of at least one herbicide that is known to interfere with AHAS activity and at a concentration or level of the herbicide that is to known to inhibit the AHAS activity of the wild-type AHASL protein. Furthermore, the AHAS activity of such a herbicide-tolerant or herbicide-resistant AHASL protein may be referred to herein as “herbicide-tolerant” or “herbicide-resistant” AHAS activity.

For the present invention, the terms “herbicide-tolerant” and “herbicide-resistant” are used interchangeable and are intended to have an equivalent meaning and an equivalent scope. Similarly, the terms “herbicide-tolerance” and “herbicide-resistance” are used interchangeable and are intended to have an equivalent meaning and an equivalent scope. Likewise, the terms “imidazolinone-resistant” and “imidazolinone-resistance” are used interchangeable and are intended to be of an equivalent meaning and an equivalent scope as the terms “imidazolinone-tolerant” and “imidazolinone-tolerance”, respectively.

The invention encompasses herbicide-resistant AHASL polynucleotides and herbicide-resistant AHASL proteins. By “herbicide-resistant AHASL polynucleotide” is intended a polynucleotide that encodes a protein comprising herbicide-resistant AHAS activity. By “herbicide-resistant AHASL protein” is intended a protein or polypeptide that comprises herbicide-resistant AHAS activity.

Further, it is recognized that a herbicide-tolerant or herbicide-resistant AHASL protein can be introduced into a plant by transforming a plant or ancestor thereof with a nucleotide sequence encoding a herbicide-tolerant or herbicide-resistant AHASL protein. Such herbicide-tolerant or herbicide-resistant AHASL proteins are encoded by the herbicide-tolerant or herbicide-resistant AHASL polynucleotides. Alternatively, a herbicide-tolerant or herbicide-resistant AHASL protein may occur in a plant as a result of a naturally occurring or induced mutation in an endogenous AHASL gene in the genome of a plant or progenitor thereof.

The present invention provides plants, plant tissues, plant cells, and host cells with increased resistance or tolerance to at least one herbicide, particularly an imidazolinone or sulfonylurea herbicide. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present invention. Typically, the effective amount of a herbicide is an amount that is routinely used in agricultural production systems to kill weeds of interest. Such an amount is known to those of ordinary skill in the art.

By “similar, wild-type, plant, plant tissue, plant cell or host cell” is intended a plant, plant tissue, plant cell, or host cell, respectively, that lacks the herbicide-resistance characteristics and/or particular polynucleotide of the invention that are disclosed herein. The use of the term “wild-type” is not, therefore, intended to imply that a plant, plant tissue, plant cell, or other host cell lacks recombinant DNA in its genome, and/or does not possess herbicide-resistant characteristics that are different from those disclosed herein.

As used herein unless clearly indicated otherwise, the term “plant” intended to mean a plant at any developmental stage, as well as any part or parts of a plant that may be attached to or separate from a whole intact plant. Such parts of a plant include, but are not limited to, organs, tissues, and cells of a plant. Examples of particular plant parts include a stem, a leaf, a root, an inflorescence, a flower, a floret, a fruit, a pedicle, a peduncle, a stamen, an anther, a stigma, a style, an ovary, a petal, a sepal, a carpel, a root tip, a root cap, a root hair, a leaf hair, a seed hair, a pollen grain, a microspore, a cotyledon, a hypocotyl, an epicotyl, xylem, phloem, parenchyma, endosperm, a companion cell, a guard cell, and any other known organs, tissues, and cells of a plant. Furthermore, it is recognized that a seed is a plant.

The plants of the present invention include both non-transgenic plants and transgenic plants. By “non-transgenic plant” is intended to mean a plant lacking recombinant DNA in its genome. By “transgenic plant” is intended to mean a plant comprising recombinant DNA in its genome. Such a transgenic plant can be produced by introducing recombinant DNA into the genome of the plant. When such recombinant DNA is incorporated into the genome of the transgenic plant, progeny of the plant can also comprise the recombinant DNA. A progeny plant that comprises at least a portion of the recombinant DNA of at least one progenitor transgenic plant is also a transgenic plant.

The present invention provides the herbicide-resistant sunflower line that is referred to herein as S4897 and progeny and derivatives thereof that comprise the herbicide-resistance characteristics of S4897. A deposit of seeds of the GM40 sunflower which is derived from sunflower line S4897 and comprises the herbicide-resistance characteristics of S4897 was made with the Patent Depository of the American Type Culture Collection (ATCC), Mansassas, Va. 20110 USA on May 17, 2005 and assigned ATCC Patent Deposit Number PTA-6716. The deposit of sunflower line GM40 was made for a term of at least 30 years and at least 5 years after the most recent request for the furnishing of a sample of the deposit is received by the ATCC. Additionally, Applicants have satisfied all the requirements of 37 C.F.R. §§1.801-1.809, including providing an indication of the viability of the sample.

The present invention further provides the herbicide-resistant sunflower line that is referred to herein as GM1606. A deposit of seeds of the sunflower GM1606 was made with the Patent Depository of the American Type Culture Collection (ATCC), Mansassas, Va. 20110 USA on May 19, 2006 and assigned ATCC Patent Deposit Number PTA-7606. The deposit of sunflower GM1606 was made for a term of at least 30 years and at least 5 years after the most recent request for the furnishing of a sample of the deposit is received by the ATCC. Additionally, Applicants have satisfied all the requirements of 37 C.F.R. §§1.801-1.809, including providing an indication of the viability of the sample.

The present invention provides herbicide-resistant sunflower plants that were produced by mutation breeding. Wild-type sunflower plants were mutagenized by exposing the plants to a mutagen, particularly a chemical mutagen, more particularly ethyl methanesulfonate (EMS). However, the present invention is not limited to herbicide-resistant sunflower plants that are produced by a mutagenesis method involving the chemical mutagen EMS. Any mutagenesis method known in the art may be used to produce the herbicide-resistant sunflower plants of the present invention. Such mutagenesis methods can involve, for example, the use of any one or more of the following mutagens: radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 2500 to 2900 nm), and chemical mutagens such as base analogues (e.g., 5-bromo-uracil), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, or acridines. Herbicide-resistant plants can also be produced by using tissue culture methods to select for plant cells comprising herbicide-resistance mutations and then regenerating herbicide-resistant plants therefrom. See, for example, U.S. Pat. Nos. 5,773,702 and 5,859,348, both of which are herein incorporated in their entirety by reference. Further details of mutation breeding can be found in “Principals of Cultivar Development” Fehr, 1993 Macmillan Publishing Company the disclosure of which is incorporated herein by reference.

Analysis of the AHASL1 gene of the sunflower plant of the S4897 and GM1606 lines revealed that the mutation that results in the substitution of a threonine for the alanine that is found at amino acid position 7 in the wild-type AHASL1 amino acid sequence for SEQ ID NO: 4. Amino acid position 7 in SEQ ID NOS: 2 and 4 corresponds to amino acid position 107 in the full-length amino acid sequence of a sunflower AHASL1 protein set forth in SEQ ID NO: 12. Thus, the present invention discloses that substituting another amino acid for the alanine at amino acid position 107, or equivalent position, in an AHASL protein can cause a sunflower plant to have enhanced resistance to a herbicide, particularly an imidazolinone and/or sulfonylurea herbicide. As disclosed in Example 6 below, the alanine at amino acid position 107 occurs within a conserved region of AHASL proteins. Similarly, amino acid substitutions in other conserved regions of AHASL proteins have been disclosed that are known to confer herbicide resistance on a plant that comprises such an AHASL protein. Accordingly, the herbicide-resistant sunflower plants of the invention include, but are not limited to those sunflower plants which comprise in their genomes at least one copy of an AHASL polynucleotide that encodes a herbicide-resistant AHASL protein that comprises a threonine at amino acid position 107 or equivalent position.

The sunflower plants of the invention further include plants that comprise, relative to the wild-type AHASL protein, a threonine or another amino acid other than alanine at amino acid position 107 or equivalent position, and one or more additional amino acid substitutions in the AHASL protein relative to the wild-type AHASL protein, wherein such a sunflower plant has increased resistance to at least one herbicide when compared to a wild-type sunflower plant. Such sunflower plants comprise AHASL proteins that comprise a threonine or another amino acid other than alanine at amino acid position 107 or equivalent position and at least one member selected from the group consisting of: an alanine, threonine, histidine, leucine, arginine, isoleucine, glutamine, or serine at amino acid position 182 or equivalent position; an isoleucine or an amino acid other than threonine at amino acid position 188 or equivalent position; an aspartate or valine at amino acid position 190 or equivalent position; a leucine at amino acid position 559 or equivalent position; and an asparagine, threonine, phenylalanine, or valine at amino acid position 638 or equivalent position.

The present invention provides AHASL proteins with amino acid substitutions at particular amino acid positions within conserved regions of the sunflower AHASL proteins disclosed herein. Furthermore, those of ordinary skill will recognize that such amino acid positions can vary depending on whether amino acids are added or removed from, for example, the N-terminal end of an amino acid sequence. Thus, the invention encompasses the amino acid substitutions at the recited position or equivalent position (e.g., “amino acid position 107 or equivalent position”). By “equivalent position” is intended to mean a position that is within the same conserved region as the exemplified amino acid position. Such conserved regions are know in the art (see Table 4 below) or can be determined by multiple sequence alignments as disclosed herein or by methods known in the art.

In addition, the present invention provides AHASL polypeptides comprising amino acid substitutions that are known to confer resistance on a plant to at least one herbicide, particularly an imidazolinone herbicide and/or a sulfonylurea herbicide. Such AHASL polypeptides include, for example, those that comprise a threonine at amino acid position 107 and at least one member selected from the group consisting of: an alanine, threonine, histidine, leucine, arginine, isoleucine, glutamine, or serine at amino acid position 182 or equivalent position; an isoleucine or another amino acid other than threonine at amino acid position 188 or equivalent position; an aspartate or valine at amino acid position 190 or equivalent position; an aspartate or valine at amino acid position 190 or equivalent position; a leucine at amino acid position 559 or equivalent position; and an asparagine, threonine, phenylalanine, or valine at amino acid position 638 or equivalent position. The invention further provides isolated polynucleotides encoding such AHASL polypeptides, as well as expression cassettes, transformation vectors, transformed host cells, transformed plants, and methods comprising such polynucleotides.

The present invention provides methods for enhancing the tolerance or resistance of a plant, plant tissue, plant cell, or other host cell to at least one herbicide that interferes with the activity of the AHAS enzyme. Preferably, such a herbicide is an imidazolinone herbicide, a sulfonylurea herbicide, a triazolopyrimidine herbicide, a pyrimidinyloxybenzoate herbicide, a sulfonylamino-carbonyltriazolinone herbicide, or mixture thereof. More preferably, such a herbicide is an imidazolinone herbicide, a sulfonylurea herbicide, or mixture thereof. For the present invention, the imidazolinone herbicides include, but are not limited to, PURSUIT® (imazethapyr), CADRE® (imazapic), RAPTOR® (imazamox), SCEPTER® (imazaquin), ASSERT® (imazethabenz), ARSENAL® (imazapyr), a derivative of any of the aforementioned herbicides, and a mixture of two or more of the aforementioned herbicides, for example, imazapyr/imazamox (ODYSSEY®). More specifically, the imidazolinone herbicide can be selected from, but is not limited to, 2-(4-isopropyl-4-methyl-5-oxo-2-imidiazolin-2-yl)-nicotinic acid, [2-(4-isopropyl)-4-][methyl-5-oxo-2-imidazolin-2-yl)-3-quinolinecarboxylic]acid, [5-ethyl-2-(4-isopropyl-] 4-methyl-5-oxo-2-imidazolin-2-yl)-nicotinic acid, 2-(4-isopropyl-4-methyl-5-oxo-2-imidazolin-2-yl)-5-(methoxymethyl)-nicotinic acid, [2-(4-isopropyl-4-methyl-5-oxo-2-] imidazolin-2-yl)-5-methylnicotinic acid, and a mixture of methyl [6-(4-isopropyl-4-] methyl-5-oxo-2-imidazolin-2-yl)-m-toluate and methyl [2-(4-isopropyl-4-methyl-5-] oxo-2-imidazolin-2-yl)-p-toluate. The use of 5-ethyl-2-(4-isopropyl-4-methyl-5-oxo-2-imidazolin-2-yl)-nicotinic acid and [2-(4-isopropyl-4-methyl-5-oxo-2-imidazolin-2-]yl)-5-(methoxymethyl)-nicotinic acid is preferred. The use of [2-(4-isopropyl-4-] methyl-5-oxo-2-imidazolin-2-yl)-5-(methoxymethyl)-nicotinic acid is particularly preferred.

For the present invention, the sulfonylurea herbicides include, but are not limited to, chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfiuon, fluzasulfuron, imazosulfuron, pyrazosulfuron ethyl, halosulfuron, azimsulfuron, cyclosulfuron, ethoxysulfuron, flazasulfuron, flupyrsulfuron methyl, foramsulfuron, iodosulfuron, oxasulfuron, mesosulfuron, prosulfuron, sulfosulfuron, trifloxysulfuron, tritosulfuron, a derivative of any of the aforementioned herbicides, and a mixture of two or more of the aforementioned herbicides. The triazolopyrimidine herbicides of the invention include, but are not limited to, cloransulam, diclosulam, florasulam, flumetsulam, metosulam, and penoxsulam. The pyrimidinyloxybenzoate herbicides of the invention include, but are not limited to, bispyribac, pyrithiobac, pyriminobac, pyribenzoxim and pyriftalid. The sulfonylamino-carbonyltriazolinone herbicides include, but are not limited to, flucarbazone and propoxycarbazone.

It is recognized that pyrimidinyloxybenzoate herbicides are closely related to the pyrimidinylthiobenzoate herbicides and are generalized under the heading of the latter name by the Weed Science Society of America. Accordingly, the herbicides of the present invention further include pyrimidinylthiobenzoate herbicides, including, but not limited to, the pyrimidinyloxybenzoate herbicides described above.

The present invention provides methods for enhancing AHAS activity in a plant comprising transforming a plant with a polynucleotide construct comprising a promoter operably linked to an AHASL1 nucleotide sequence of the invention. The methods involve introducing a polynucleotide construct of the invention into at least one plant cell and regenerating a transformed plant therefrom. The methods involve the use of a promoter that is capable of driving gene expression in a plant cell. Preferably, such a promoter is a constitutive promoter or a tissue-preferred promoter. The methods find use in enhancing or increasing the resistance of a plant to at least one herbicide that interferes with the catalytic activity of the AHAS enzyme, particularly an imidazolinone herbicide.

The present invention provides expression cassettes for expressing the polynucleotides of the invention in plants, plant tissues, plant cells, and other host cells. The expression cassettes comprise a promoter expressible in the plant, plant tissue, plant cell, or other host cells of interest operably linked to a polynucleotide of the invention that comprises a nucleotide sequence encoding either a full-length (i.e. including the chloroplast transit peptide) or mature AHASL1 protein (i.e. without the chloroplast transit peptide). If expression is desired in the plastids or chloroplasts of plants or plant cells, the expression cassette may also comprise an operably linked chloroplast-targeting sequence that encodes a chloroplast transit peptide.

The expression cassettes of the invention find use in a method for enhancing the herbicide tolerance of a plant or a host cell. The method involves transforming the plant or host cell with an expression cassette of the invention, wherein the expression cassette comprises a promoter that is expressible in the plant or host cell of interest and the promoter is operably linked to a polynucleotide of the invention that comprises a nucleotide sequence encoding an imidazolinone-resistant AHASL protein of the invention.

The use of the term “polynucleotide constructs” herein is not intended to limit the present invention to polynucleotide constructs comprising DNA. Those of ordinary skill in the art will recognize that polynucleotide constructs, particularly polynucleotides and oligonucleotides, comprised of ribonucleotides and combinations of ribonucleotides and deoxyribonucleotides may also be employed in the methods disclosed herein. Thus, the polynucleotide constructs of the present invention encompass all polynucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The polynucleotide constructs of the invention also encompass all forms of polynucleotide constructs including, but not limited to, single-stranded forms, double-stranded forms, hairpins, stem-and-loop structures, and the like. Furthermore, it is understood by those of ordinary skill in the art that each nucleotide sequences disclosed herein also encompasses the complement of that exemplified nucleotide sequence.

Furthermore, it is recognized that the methods of the invention may employ a polynucleotide construct that is capable of directing, in a transformed plant, the expression of at least one protein, or at least one RNA, such as, for example, an antisense RNA that is complementary to at least a portion of an mRNA. Typically such a polynucleotide construct is comprised of a coding sequence for a protein or an RNA operably linked to 5′ and 3′ transcriptional regulatory regions. Alternatively, it is also recognized that the methods of the invention may employ a polynucleotide construct that is not capable of directing, in a transformed plant, the expression of a protein or an RNA.

Further, it is recognized that, for expression of a polynucleotide of the invention in a host cell of interest, the polynucleotide is typically operably linked to a promoter that is capable of driving gene expression in the host cell of interest. The methods of the invention for expressing the polynucleotides in host cells do not depend on particular promoter. The methods encompass the use of any promoter that is known in the art and that is capable of driving gene expression in the host cell of interest.

The present invention encompasses AHASL polynucleotide molecules and fragments and variants thereof. Polynucleotide molecules that are fragments of these nucleotide sequences are also encompassed by the present invention. By “fragment” is intended a portion of the nucleotide sequence encoding an AHASL protein of the invention. A fragment of an AHASL nucleotide sequence of the invention may encode a biologically active portion of an AHASL protein, or it may be a fragment that can be used as a hybridization probe or PCR primer using methods disclosed below. A biologically active portion of an AHASL protein can be prepared by isolating a portion of one of the AHASL nucleotide sequences of the invention, expressing the encoded portion of the AHASL protein (e.g., by recombinant expression in vitro), and assessing the activity of the encoded portion of the AHASL1 protein. Polynucleotide molecules that are fragments of an AHASL nucleotide sequence comprise at least about 15, 20, 50, 75, 100, 200, 300, 350, 400, 450, 500, 550, 600, 650, 700, 750, 800, 850, 900, 950, 1000, 1050, or 1100 nucleotides, or up to the number of nucleotides present in a full-length nucleotide sequence disclosed herein (for example, 1178 nucleotides for both SEQ ID NOS: 1 and 3) depending upon the intended use.

A fragment of an AHASL nucleotide sequence that encodes a biologically active portion of an AHASL protein of the invention will encode at least about 15, 25, 30, 50, 75, 100, 125, 150, 175, 200, 250, 300, or 350 contiguous amino acids, or up to the total number of amino acids present in a full-length AHASL1 protein of the invention (for example, 392 amino acids for both SEQ ID NOS: 2 and 4). Fragments of an AHASL1 nucleotide sequence that are useful as hybridization probes for PCR primers generally need not encode a biologically active portion of an AHASL1 protein.

Polynucleotide molecules that are variants of the nucleotide sequences disclosed herein are also encompassed by the present invention. “Variants” of the AHASL nucleotide sequences of the invention include those sequences that encode the AHASL proteins disclosed herein but that differ conservatively because of the degeneracy of the genetic code. These naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the AHASL1 protein disclosed in the present invention as discussed below. Generally, nucleotide sequence variants of the invention will have at least about 70%, 75%, 80%, 85%, 90%, 91%, 92%, 93%, 94%, 95%, 96%, 97%, 98%, or 99% identity to a particular nucleotide sequence disclosed herein. A variant AHASL nucleotide sequence will encode an AHASL protein, respectively, that has an amino acid sequence having at least about 70%, 75%, 80%, 85%, 90%, 91%, 92%, 93%, 94%, 95%, 96%, 97%, 98%, or 99% identity to the amino acid sequence of an AHASL protein disclosed herein.

In addition, the skilled artisan will further appreciate that changes can be introduced by mutation into the nucleotide sequences of the invention thereby leading to changes in the amino acid sequence of the encoded AHASL proteins without altering the biological activity of the AHASL proteins. Thus, an isolated polynucleotide molecule encoding an AHASL protein having a sequence that differs from that of SEQ ID NOS: 1 or 3, respectively, can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, preferably, conservative amino acid substitutions may be made at one or more predicted, preferably nonessential amino acid residues. A “nonessential” amino acid residue is a residue that can be altered from the wild-type sequence of an AHASL protein (e.g., the sequence of SEQ ID NOS: 2 and 4, respectively) without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif.

The proteins of the invention may be altered in various ways including amino acid substitutions, deletions, truncations, and insertions. Methods for such manipulations are generally known in the art. For example, amino acid sequence variants of the AHASL proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Alternatively, variant AHASL nucleotide sequences can be made by introducing mutations randomly along all or part of an AHASL coding sequence, such as by saturation mutagenesis, and the resultant mutants can be screened for AHAS activity to identify mutants that retain AHAS activity, including herbicide-resistant AHAS activity. Following mutagenesis, the encoded protein can be expressed recombinantly, and the activity of the protein can be determined using standard assay techniques.

Thus, the nucleotide sequences of the invention include the sequences disclosed herein as well as fragments and variants thereof. The AHASL nucleotide sequences of the invention, and fragments and variants thereof, can be used as probes and/or primers to identify and/or clone AHASL homologues in other plants. Such probes can be used to detect transcripts or genomic sequences encoding the same or identical proteins.

In this manner, methods such as PCR, hybridization, and the like can be used to identify such sequences having substantial identity to the sequences of the invention. See, for example, Sambrook et al. (1989) Molecular Cloning: Laboratory Manual (2d ed., Cold Spring Harbor Laboratory Press, Plainview, N.Y.) and Innis, et al. (1990) PCR Protocols: A Guide to Methods and Applications (Academic Press, NY). AHASL nucleotide sequences isolated based on their sequence identity to the AHASL1 nucleotide sequences set forth herein or to fragments and variants thereof are encompassed by the present invention.

In a hybridization method, all or part of a known AHASL nucleotide sequence can be used to screen cDNA or genomic libraries. Methods for construction of such cDNA and genomic libraries are generally known in the art and are disclosed in Sambrook et al. (1989) Molecular Cloning: A Laboratory Manual (2d ed., Cold Spring Harbor Laboratory Press, Plainview, N.Y.). The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known AHASL nucleotide sequence disclosed herein. Degenerate primers designed on the basis of conserved nucleotides or amino acid residues in a known AHASL nucleotide sequence or encoded amino acid sequence can additionally be used. The probe typically comprises a region of nucleotide sequence that hybridizes under stringent conditions to at least about 12, preferably about 25, more preferably about 50, 75, 100, 125, 150, 175, 200, 250, 300, 350, 400, 500, 600, 700, 800, 900, 1000, 1100, or 1178 consecutive nucleotides of an AHASL nucleotide sequence of the invention or a fragment or variant thereof. Preparation of probes for hybridization is generally known in the art and is disclosed in Sambrook et al. (1989) Molecular Cloning: A Laboratory Manual (2d ed., Cold Spring Harbor Laboratory Press, Plainview, N.Y.), herein incorporated by reference.

For example, the entire AHASL sequence disclosed herein, or one or more portions thereof, may be used as a probe capable of specifically hybridizing to corresponding AHASL sequences and messenger RNAs. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

It is recognized that the polynucleotide molecules and proteins of the invention encompass polynucleotide molecules and proteins comprising a nucleotide or an amino acid sequence that is sufficiently identical to the nucleotide sequence of SEQ ID NOS: 1 and/or 3, or to the amino acid sequence of SEQ ID NOS: 2 and/or 4. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 95%, or 98% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and)(BLAST programs of Altschul et al. (1990) J. Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22(22):4673-4680, 1994) using the program AlignX included in the software package Vector NTI Suite Version 7 (InforMax, Inc., Bethesda, Md., USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by AlignX in the software package Vector NTI Suite Version 7.

The AHASL nucleotide sequences of the invention include both the naturally occurring sequences as well as mutant forms, particularly mutant forms that encode AHASL proteins comprising herbicide-resistant AHAS activity. Likewise, the proteins of the invention encompass both naturally occurring proteins as well as variations and modified forms thereof. Such variants will continue to possess the desired AHAS activity. Obviously, the mutations that will be made in the DNA encoding the variant must not place the sequence out of reading frame and preferably will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by AHAS activity assays. See, for example, Singh et al. (1988) Anal. Biochem. 171:173-179, herein incorporated by reference.

Variant nucleotide sequences and proteins also encompass sequences and proteins derived from a mutagenic and recombinogenic procedure such as DNA shuffling. With such a procedure, one or more different AHASL coding sequences can be manipulated to create a new AHASL protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the AHASL gene of the invention and other known AHASL genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The nucleotide sequences of the invention can be used to isolate corresponding sequences from other organisms, particularly other plants, more particularly other dicots. In this manner, methods such as PCR, hybridization, and the like can be used to identify such sequences based on their sequence homology to the sequences set forth herein. Sequences isolated based on their sequence identity to the entire AHASL polynucleotide sequences set forth herein or to fragments thereof are encompassed by the present invention. Thus, isolated polynucleotide sequences that encode for an AHASL protein and which hybridize under stringent conditions to the sequence disclosed herein, or to fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The AHASL polynucleotide sequences of the invention are provided in expression cassettes for expression in the plant of interest. The cassette will include 5′ and 3′ regulatory sequences operably linked to an AHASL polynucleotide sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the AHASL polynucleotide sequence to be under the transcriptional regulation of the regulatory regions. The expression cassette may additionally contain selectable marker genes.

The expression cassette will include in the 5′-3′ direction of transcription, a transcriptional and translational initiation region (i.e., a promoter), an AHASL polynucleotide sequence of the invention, and a transcriptional and translational termination region (i.e., termination region) functional in plants. The promoter may be native or analogous, or foreign or heterologous, to the plant host and/or to the AHASL polynucleotide sequence of the invention. Additionally, the promoter may be the natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the AHASL polynucleotide sequence of the invention, it is intended that the promoter is not the native or naturally occurring promoter for the operably linked AHASL polynucleotide sequence of the invention. As used herein, a chimeric gene comprises a coding sequence operably linked to a transcription initiation region that is heterologous to the coding sequence.

While it may be preferable to express the AHASL polynucleotides of the invention using heterologous promoters, the native promoter sequences may be used. Such constructs would change expression levels of the AHASL protein in the plant or plant cell. Thus, the phenotype of the plant or plant cell is altered.

The termination region may be native with the transcriptional initiation region, may be native with the operably linked AHASL sequence of interest, may be native with the plant host, or may be derived from another source (i.e., foreign or heterologous to the promoter, the AHASL polynucleotide sequence of interest, the plant host, or any combination thereof). Convenient termination regions are available from the Ti-plasmid of A. tumefaciens, such as the octopine synthase and nopaline synthase termination regions. See also Guerineau et al. (1991)Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

Nucleotide sequences for enhancing gene expression can also be used in the plant expression vectors. These include the introns of the maize AdhI, intron1 gene (Callis et al. Genes and Development 1:1183-1200, 1987), and leader sequences, (W-sequence) from the Tobacco Mosaic virus (TMV), Maize Chlorotic Mottle Virus and Alfalfa Mosaic Virus (Gallie et al. Nucleic Acid Res. 15:8693-8711, 1987 and Skuzeski et al. Plant Mol. Biol. 15:65-79, 1990). The first intron from the shrunken-1 locus of maize, has been shown to increase expression of genes in chimeric gene constructs. U.S. Pat. Nos. 5,424,412 and 5,593,874 disclose the use of specific introns in gene expression constructs, and Gallie et al. (Plant Physiol. 106:929-939, 1994) also have shown that introns are useful for regulating gene expression on a tissue specific basis. To further enhance or to optimize AHAS large subunit gene expression, the plant expression vectors of the invention may also contain DNA sequences containing matrix attachment regions (MARs). Plant cells transformed with such modified expression systems, then, may exhibit overexpression or constitutive expression of a nucleotide sequence of the invention.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced AHASL1 expression within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6): 1129-1138; Matsuoka et al. (1993) Proc Natl. Acad. Sci. USA 90(20): 9586-9590; and Guevara-Garcia et al. (1993) Plant J. 4(3):495-505. Such promoters can be modified, if necessary, for weak expression.

In one embodiment, the nucleic acids of interest are targeted to the chloroplast for expression. In this manner, where the nucleic acid of interest is not directly inserted into the chloroplast, the expression cassette will additionally contain a chloroplast-targeting sequence comprising a nucleotide sequence that encodes a chloroplast transit peptide to direct the gene product of interest to the chloroplasts. Such transit peptides are known in the art. With respect to chloroplast-targeting sequences, “operably linked” means that the nucleic acid sequence encoding a transit peptide (i.e., the chloroplast-targeting sequence) is linked to the AHASL polynucleotide of the invention such that the two sequences are contiguous and in the same reading frame. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481. While the AHASL proteins of the invention include a native chloroplast transit peptide, any chloroplast transit peptide known in the art can be fused to the amino acid sequence of a mature AHASL protein of the invention by operably linking a choloroplast-targeting sequence to the 5′-end of a nucleotide sequence encoding a mature AHASL protein of the invention.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(10:6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999), See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Methods for transformation of chloroplasts are known in the art. See, for example, Svab et al. (1990) Proc. Natl. Acad. Sci. USA 87:8526-8530; Svab and Maliga (1993) Proc. Natl. Acad Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

As disclosed herein, the AHASL nucleotide sequences of the invention find use in enhancing the herbicide tolerance of plants that comprise in their genomes a gene encoding a herbicide-tolerant AHASL protein. Such a gene may be an endogenous gene or a transgene. Additionally, in certain embodiments, the nucleic acid sequences of the present invention can be stacked with any combination of polynucleotide sequences of interest in order to create plants with a desired phenotype. For example, the polynucleotides of the present invention may be stacked with any other polynucleotides encoding polypeptides having pesticidal and/or insecticidal activity, such as, for example, the Bacillus thuringiensis toxin proteins (described in U.S. Pat. Nos. 5,366,892; 5,747,450; 5,737,514; 5,723,756; 5,593,881; and Geiser et al. (1986) Gene 48:109). The combinations generated can also include multiple copies of any one of the polynucleotides of interest.

It is recognized that with these nucleotide sequences, antisense constructions, complementary to at least a portion of the messenger RNA (mRNA) for the AHASL polynucleotide sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See, U.S. Pat. Nos. 5,283,184 and 5,034,323; herein incorporated by reference.

While the herbicide-resistant AHASL1 polynucleotides of the invention find use as selectable masker genes for plant transformation, the expression cassettes of the invention can include another selectable marker gene for the selection of transformed cells. Selectable marker genes, including those of the present invention, are utilized for the selection of transformed cells or tissues. Marker genes include, but are not limited to, genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Flu et al. (1987) Cell 48:555-566; Brown et al. (1987) Cell 49:603-612; Figge et al. (1988) Cell 52:713-722; Deuschle et al. (1989) Proc. Natl. Acad Aci. USA 86:5400-5404; Fuerst et at (1989) Proc. Natl. Acad Sci. USA 86:2549-2553; Deuschle et al. (1990) Science 248:480-483; Gossen (1993) Ph.D. Thesis, University of Heidelberg; Reines et al. (1993) Proc. Natl. Acad Sci. USA 90:1917-1921; Labow et al. (1990)Mol. Cell. Biol. 10:3343-3356; Zambretti et al. (1992) Proc. Natl. Acad. Sci. USA 89:3952-3956; Baim et al. (1991) Proc. Natl. Acad Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et at (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

The isolated polynucleotide molecules comprising nucleotide sequence that encode the AHASL proteins of the invention can be used in vectors to transform plants so that the plants created have enhanced resistant to herbicides, particularly imidazolinone herbicides. The isolated AHASL polynucleotide molecules of the invention can be used in vectors alone or in combination with a nucleotide sequence encoding the small subunit of the AHAS (AHASS) enzyme in conferring herbicide resistance in plants. See, U.S. Pat. No. 6,348,643; which is herein incorporated by reference.

The invention also relates to a plant expression vector comprising a promoter that drives expression in a plant operably linked to an isolated polynucleotide molecule of the invention. The isolated polynucleotide molecule comprises a nucleotide sequence encoding an AHASL protein, particularly an AHASL protein comprising an amino sequence that is set forth in SEQ ID NO: 2 or 4, or a functional fragment and variant thereof. The plant expression vector of the invention does not depend on a particular promoter, only that such a promoter is capable of driving gene expression in a plant cell. Preferred promoters include constitutive promoters and tissue-preferred promoters.

The transformation vectors of the invention can be used to produce plants transformed with a gene of interest. The transformation vector will comprise a selectable marker gene of the invention and a gene of interest to be introduced and typically expressed in the transformed plant. Such a selectable marker gene comprises a herbicide-resistant AHASL polynucleotide of the invention operably linked to a promoter that drives expression in a host cell. For use in plants and plant cells, the transformation vector comprises a selectable marker gene comprising a herbicide-resistant AHASL polynucleotide of the invention operably linked to a promoter that drives expression in a plant cell.

The genes of interest of the invention vary depending on the desired outcome. For example, various changes in phenotype can be of interest including modifying the fatty acid composition in a plant, altering the amino acid content of a plant, altering a plant's insect and/or pathogen defense mechanisms, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

In one embodiment of the invention, the genes of interest include insect resistance genes such as, for example, Bacillus thuringiensis toxin protein genes (U.S. Pat. Nos. 5,366,892; 5,747,450; 5,736,514; 5,723,756; 5,593,881; and Geiser et al. (1986) Gene 48:109).

The AHASL proteins or polypeptides of the invention can be purified from, for example, sunflower plants and can be used in compositions. Also, an isolated polynucleotide molecule encoding an AHASL protein of the invention can be used to express an AHASL protein of the invention in a microbe such as E. coli or a yeast. The expressed AHASL protein can be purified from extracts of E. coli or yeast by any method known to those of ordinary skill in the art.

The invention also relates to a method for creating a transgenic plant that is resistant to herbicides, comprising transforming a plant with a plant expression vector comprising a promoter that drives expression in a plant operably linked to an isolated polynucleotide molecule of the invention. The isolated polynucleotide molecule comprises a nucleotide sequence encoding an AHASL protein of the invention, particularly an AHASL protein comprising: an amino sequence that is set forth in SEQ ID NO: 2, an amino acid sequence encoded by SEQ ID NO: 1, or a functional fragment and variant of said amino acid sequences.

The invention also relates to the non-transgenic sunflower plants, transgenic plants produced by the methods of the invention, and progeny and other descendants of such non-transgenic and transgenic plants, which plants exhibit enhanced or increased resistance to herbicides that interfere with the AHAS enzyme, particularly imidazolinone and sulfonylurea herbicides.

The AHASL polynucleotides of the invention, particularly those encoding herbicide-resistant AHASL proteins, find use in methods for enhancing the resistance of herbicide-tolerant plants. In one embodiment of the invention, the herbicide-tolerant plants comprise a herbicide-tolerant or herbicide-resistant AHASL protein. The herbicide-tolerant plants include both plants transformed with a herbicide-tolerant AHASL nucleotide sequences and plants that comprise in their genomes an endogenous gene that encodes a herbicide-tolerant AHASL protein. Nucleotide sequences encoding herbicide-tolerant AHASL proteins and herbicide-tolerant plants comprising an endogenous gene that encodes a herbicide-tolerant AHASL protein include the polynucleotides and plants of the present invention and those that are known in the art. See, for example, U.S. Pat. Nos. 5,013,659, 5,731,180, 5,767,361, 5,545,822, 5,736,629, 5,773,703, 5,773,704, 5,952,553 and 6,274,796; all of which are herein incorporated by reference. Such methods for enhancing the resistance of herbicide-tolerant plants comprise transforming a herbicide-tolerant plant with at least one polynucleotide construction comprising a promoter that drives expression in a plant cell that is operably linked to a herbicide-resistant AHASL polynucleotide of the invention, particularly the polynucleotide encoding a herbicide-resistant AHASL protein set forth in SEQ ID NO: 1, polynucleotides encoding the amino acid sequence set forth in SEQ ID NO: 2, and fragments and variants said polynucleotides that encode polypeptides comprising herbicide-resistant AHAS activity.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.—Plant; 29P:119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

The methods of the invention involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed. In an embodiment of the invention, an AHASL1 nucleotide sequence is operably linked to a plant promoter that is known for high-level expression in a plant cell, and this construct is then introduced into a plant that is susceptible to an imidazolinone herbicide and a transformed plant is regenerated. The transformed plant is tolerant to exposure to a level of an imidazolinone herbicide that would kill or significantly injure an untransformed plant. This method can be applied to any plant species; however, it is most beneficial when applied to crop plants.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991)Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Set. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et at (1998) Theor. Appl. Genet. 96:319-324 (soybean); Datta et al. (1990) Biotechnology 8:736-740 (rice); Klein et al. (1988) Proc. Natl. Acad Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. It is recognized that the an AHASL protein of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct of the invention, for example, an expression cassette of the invention, stably incorporated into their genome.

The present invention may be used for transformation of any plant species, including, but not limited to, monocots and dicots. Examples of plant species of interest include, but are not limited to, corn or maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum, T. Turgidum ssp. durum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus carica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. Preferably, plants of the present invention are crop plants (for example, sunflower, Brassica sp., cotton, sugar beet, soybean, peanut, alfalfa, safflower, tobacco, corn, rice, wheat, rye, barley triticale, sorghum, millet, etc.).

The herbicide-resistant plants of the invention find use in methods for controlling weeds. Thus, the present invention further provides a method for controlling weeds in the vicinity of a herbicide-resistant plant of the invention. The method comprises applying an effective amount of a herbicide to the weeds and to the herbicide-resistant plant, wherein the plant has increased resistance to at least one herbicide, particularly an imidazolinone or sulfonylurea herbicide, when compared to a wild-type plant. In such a method for controlling weeds, the herbicide-resistant plants of the invention are preferably crop plants, including, but not limited to, sunflower, alfalfa, Brassica sp., soybean, cotton, safflower, peanut, tobacco, tomato, potato, wheat, rice, maize, sorghum, barley, rye, millet, and sorghum.

By providing plants having increased resistance to herbicides, particularly imidazolinone and sulfonylurea herbicides, a wide variety of formulations can be employed for protecting plants from weeds, so as to enhance plant growth and reduce competition for nutrients. A herbicide can be used by itself for pre-emergence, post-emergence, pre-planting and at planting control of weeds in areas surrounding the plants described herein or an imidazolinone herbicide formulation can be used that contains other additives. The herbicide can also be used as a seed treatment. Additives found in an imidazolinone or sulfonylurea herbicide formulation include other herbicides, detergents, adjuvants, spreading agents, sticking agents, stabilizing agents, or the like. The herbicide formulation can be a wet or dry preparation and can include, but is not limited to, flowable powders, emulsifiable concentrates and liquid concentrates. The herbicide and herbicide formulations can be applied in accordance with conventional methods, for example, by spraying, irrigation, dusting, or the like.

The present invention provides non-transgenic and transgenic seeds with increased tolerance to at least one herbicide, particularly an AHAS-inhibiting herbicide, more particularly imidazolinone and sulfonylurea herbicides. Such seeds include, for example, non-transgenic sunflower seeds comprising the herbicide-tolerance characteristics of the sunflower plant S4897, the sunflower plant GM40, the sunflower plant GM1606, the sunflower plant with ATCC Patent Deposit Number PTA-6716, or the sunflower plant with ATCC Patent Deposit Number PTA-7606, and transgenic seeds comprising a polynucleotide molecule of the invention that encodes a herbicide-resistant AHASL protein.

The present invention provides methods for producing a herbicide-resistant plant, particularly a herbicide-resistant sunflower plant, through conventional plant breeding involving sexual reproduction. The methods comprise crossing a first plant that is resistant to a herbicide to a second plant that is not resistant to the herbicide. The first plant can be any of the herbicide resistant plants of the present invention including, for example, transgenic plants comprising at least one of the polynucleotide molecules of the present invention that encode a herbicide resistant AHASL protein and non-transgenic sunflower plants that comprise the herbicide-tolerance characteristics of the sunflower plant S4897, the sunflower plant GM40, the sunflower plant GM1606, the sunflower plant with ATCC Patent Deposit Number PTA-6716, or the sunflower plant with ATCC Patent Deposit Number PTA-7606. The second plant can be any plant that is capable of producing viable progeny plants (i.e., seeds) when crossed with the first plant. Typically, but not necessarily, the first and second plants are of the same species. The methods of the invention can further involve one or more generations of backcrossing the progeny plants of the first cross to a plant of the same line or genotype as either the first or second plant. Alternatively, the progeny of the first cross or any subsequent cross can be crossed to a third plant that is of a different line or genotype than either the first or second plant. The methods of the invention can additionally involve selecting plants that comprise the herbicide tolerance characteristics of the first plant.

The present invention further provides methods for increasing the herbicide-resistance of a plant, particularly a herbicide-resistant sunflower plant, through conventional plant breeding involving sexual reproduction. The methods comprise crossing a first plant that is resistant to a herbicide to a second plant that may or may not be resistant to the herbicide or may be resistant to different herbicide or herbicides than the first plant. The first plant can be any of the herbicide resistant plants of the present invention including, for example, transgenic plants comprising at least one of the polynucleotide molecules of the present invention that encode a herbicide-resistant AHASL protein and non-transgenic sunflower plants that comprise the herbicide-tolerance characteristics of the sunflower plant S4897, the sunflower plant GM40, the sunflower plant GM1606, the sunflower plant with ATCC Patent Deposit Number PTA-6716, or the sunflower plant with ATCC Patent Deposit Number PTA-7606. The second plant can be any plant that is capable of producing viable progeny plants (i.e., seeds) when crossed with the first plant. Typically, but not necessarily, the first and second plants are of the same species. The progeny plants produced by this method of the present invention have increased resistance to a herbicide when compared to either the first or second plant or both. When the first and second plants are resistant to different herbicides, the progeny plants will have the combined herbicide tolerance characteristics of the first and second plants. The methods of the invention can further involve one or more generations of backcrossing the progeny plants of the first cross to a plant of the same line or genotype as either the first or second plant. Alternatively, the progeny of the first cross or any subsequent cross can be crossed to a third plant that is of a different line or genotype than either the first or second plant. The methods of the invention can additionally involve selecting plants that comprise the herbicide tolerance characteristics of the first plant, the second plant, or both the first and the second plant.

The plants of the present invention can be transgenic or non-transgenic. An example of a non-transgenic sunflower plant having increased resistance to imidazolinone is the sunflower plant sunflower plant S4897, the sunflower plant GM40, the sunflower plant GM1606, the sunflower plant with ATCC Patent Deposit Number PTA-6716, or the sunflower plant with ATCC Patent Deposit Number PTA-7606; or mutant, recombinant, or a genetically engineered derivative of the sunflower plant S4897, the sunflower plant GM40, the sunflower plant GM1606, the sunflower plant with ATCC Patent Deposit Number PTA-6716, or the sunflower plant with ATCC Patent Deposit Number PTA-7606; or of any progeny of the sunflower plant S4897, the sunflower plant GM40, the sunflower plant GM1606, the sunflower plant with ATCC Patent Deposit Number PTA-6716, or the sunflower plant with ATCC Patent Deposit Number PTA-7606; or a plant that is a progeny of any of these plants; or a plant that comprises the herbicide tolerance characteristics of the sunflower plant S4897, the sunflower plant GM40, the sunflower plant GM1606, the sunflower plant with ATCC Patent Deposit Number PTA-6716, or the sunflower plant with ATCC Patent Deposit Number PTA-7606.

The present invention also provides plants, plant organs, plant tissues, plant cells, seeds, and non-human host cells that are transformed with the at least one polynucleotide molecule, expression cassette, or transformation vector of the invention. Such transformed plants, plant organs, plant tissues, plant cells, seeds, and non-human host cells have enhanced tolerance or resistance to at least one herbicide, at levels of the herbicide that kill or inhibit the growth of an untransformed plant, plant tissue, plant cell, or non-human host cell, respectively. Preferably, the transformed plants, plant tissues, plant cells, and seeds of the invention are Arabidopsis thaliana and crop plants.

The present invention provides methods that involve the use of at least one AHAS-inhibiting herbicide selected from the group consisting of imidazolinone herbicides, sulfonylurea herbicides, triazolopyrimidine herbicides, pyrimidinyloxybenzoate herbicides, sulfonylamino-carbonyltriazolinone herbicides, and mixtures thereof. In these methods, the AHAS-inhibiting herbicide can be applied by any method known in the art including, but not limited to, seed treatment, soil treatment, and foliar treatment.

Prior to application, the AHAS-inhibiting herbicide can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.

Examples of suitable carriers are ground natural minerals (for example kaolins, clays, talc, chalk) and ground synthetic minerals (for example highly disperse silica, silicates).

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

Suitable preservatives are for example Dichlorophen and enzylalkoholhemiformal.

Seed Treatment formulations may additionally comprise binders and optionally colorants.

Binders can be added to improve the adhesion of the active materials on the seeds after treatment. Suitable binders are block copolymers EO/PO surfactants but also polyvinylalcoholsl, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

An example of a suitable gelling agent is carrageen (Satiagel®).

Powders, materials for spreading, and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the AHAS-inhibiting herbicide. In this case, the AHAS-inhibiting herbicides are employed in a purity of from 90% to 100% by weight, preferably 95% to 100% by weight (according to NMR spectrum). For seed treatment purposes, respective formulations can be diluted 2-10 fold leading to concentrations in the ready to use preparations of 0.01 to 60% by weight active compound by weight, preferably 0.1 to 40% by weight.

The AHAS-inhibiting herbicide can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the AHAS-inhibiting herbicide according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The AHAS-inhibiting herbicide may also be used successfully in the ultra-low-volume process (ULV), it being possible to apply formulations comprising over 95% by weight of active compound, or even to apply the active compound without additives.

The following are examples of formulations:
1. Products for dilution with water for foliar applications. For seed treatment purposes, such products may be applied to the seed diluted or undiluted.
A) Water-soluble concentrates (SL, LS)
Ten parts by weight of the AHAS-inhibiting herbicide are dissolved in 90 parts by weight of water or a water-soluble solvent. As an alternative, welters or other auxiliaries are added. The AHAS-inhibiting herbicide dissolves upon dilution with water, whereby a formulation with 10% (w/w) of AHAS-inhibiting herbicide is obtained.
B) Dispersible concentrates (DC)
Twenty parts by weight of the AHAS-inhibiting herbicide are dissolved in 70 parts by weight of cyclohexanone with addition of 10 parts by weight of a dispersant, for example polyvinylpyrrolidone. Dilution with water gives a dispersion, whereby a formulation with 20% (w/w) of AHAS-inhibiting herbicide is obtained.
C) Emulsifiable concentrates (EC)
Fifteen parts by weight of the AHAS-inhibiting herbicide are dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of AHAS-inhibiting herbicide is obtained.
D) Emulsions (EW, EO, ES)
Twenty-five parts by weight of the AHAS-inhibiting herbicide are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of AHAS-inhibiting herbicide is obtained.
E) Suspensions (SC, OD, FS)
In an agitated ball mill, 20 parts by weight of the AHAS-inhibiting herbicide are comminuted with addition of 10 parts by weight of dispersants, wetters and 70 parts by weight of water or of an organic solvent to give a fine AHAS-inhibiting herbicide suspension. Dilution with water gives a stable suspension of the AHAS-inhibiting herbicide, whereby a formulation with 20% (w/w) of AHAS-inhibiting herbicide is obtained.
F) Water-dispersible granules and water-soluble granules (WG, SG)
Fifty parts by weight of the AHAS-inhibiting herbicide are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the AHAS-inhibiting herbicide, whereby a formulation with 50% (w/w) of AHAS-inhibiting herbicide is obtained.
G) Water-dispersible powders and water-soluble powders (WP, SP, SS, WS)
Seventy-five parts by weight of the AHAS-inhibiting herbicide are ground in a rotor-stator mill with addition of 25 parts by weight of dispersants, wetters and silica gel. Dilution with water gives a stable dispersion or solution of the AHAS-inhibiting herbicide, whereby a formulation with 75% (w/w) of AHAS-inhibiting herbicide is obtained.
I) Gel-Formulation (GF)
In an agitated ball mill, 20 parts by weight of the AHAS-inhibiting herbicide are comminuted with addition of 10 parts by weight of dispersants, 1 part by weight of a gelling agent wetters and 70 parts by weight of water or of an organic solvent to give a fine AHAS-inhibiting herbicide suspension. Dilution with water gives a stable suspension of the AHAS-inhibiting herbicide, whereby a formulation with 20% (w/w) of AHAS-inhibiting herbicide is obtained. This gel formulation is suitable for us as a seed treatment.
2. Products to be applied undiluted for foliar applications. For seed treatment purposes, such products may be applied to the seed diluted.
A) Dustable powders (DP, DS)
Five parts by weight of the AHAS-inhibiting herbicide are ground finely and mixed intimately with 95 parts by weight of finely divided kaolin. This gives a dustable product having 5% (w/w) of AHAS-inhibiting herbicide.
B) Granules (GR, FG, GG, MG)
One-half part by weight of the AHAS-inhibiting herbicide is ground finely and associated with 95.5 parts by weight of carriers, whereby a formulation with 0.5% (w/w) of AHAS-inhibiting herbicide is obtained. Current methods are extrusion, spray-drying or the fluidized bed. This gives granules to be applied undiluted for foliar use.

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

The present invention non-transgenic and transgenic seeds of the herbicide-resistant plants of the present invention. Such seeds include, for example, non-transgenic sunflower seeds comprising the herbicide-tolerance characteristics of the plant with NCIMB Accession Number NCIMB 41262, and transgenic seeds comprising a polynucleotide molecule of the invention that encodes an IMI protein.

For seed treatment, seeds of the herbicide resistant plants according of the present invention are treated with herbicides, preferably herbicides selected from the group consisting of AHAS-inhibiting herbicides such as amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid, pyrithiobac, and mixtures thereof, or with a formulation comprising a AHAS-inhibiting herbicide.

The term seed treatment comprises all suitable seed treatment techniques known in the art, such as seed dressing, seed coating, seed dusting, seed soaking, and seed pelleting.

In accordance with one variant of the present invention, a further subject of the invention is a method of treating soil by the application, in particular into the seed drill: either of a granular formulation containing the AHAS-inhibiting herbicide as a composition/formulation (e.g. a granular formulation, with optionally one or more solid or liquid, agriculturally acceptable carriers and/or optionally with one or more agriculturally acceptable surfactants. This method is advantageously employed, for example, in seedbeds of cereals, maize, cotton, and sunflower.

The present invention also comprises seeds coated with or containing with a seed treatment formulation comprising at least one AHAS-inhibiting herbicide selected from the group consisting of amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

The seed treatment application with the AHAS-inhibiting herbicide or with a formulation comprising the AHAS-inhibiting herbicide is carried out by spraying or dusting the seeds before sowing of the plants and before emergence of the plants.

In the treatment of seeds, the corresponding formulations are applied by treating the seeds with an effective amount of the AHAS-inhibiting herbicide or a formulation comprising the AHAS-inhibiting herbicide. Herein, the application rates are generally from 0.1 g to 10 kg of the a.i. (or of the mixture of a.i. or of the formulation) per 100 kg of seed, preferably from 1 g to 5 kg per 100 kg of seed, in particular from 1 g to 2.5 kg per 100 kg of seed. For specific crops such as lettuce the rate can be higher.

The present invention provides a method for combating undesired vegetation or controlling weeds comprising contacting the seeds of the resistant plants according to the present invention before sowing and/or after pregermination with an AHAS-inhibiting herbicide. The method can further comprise sowing the seeds, for example, in soil in a field or in a potting medium in greenhouse. The method finds particular use in combating undesired vegetation or controlling weeds in the immediate vicinity of the seed.

The control of undesired vegetation is understood as meaning the killing of weeds and/or otherwise retarding or inhibiting the normal growth of the weeds. Weeds, in the broadest sense, are understood as meaning all those plants which grow in locations where they are undesired.

The weeds of the present invention include, for example, dicotyledonous and monocotyledonous weeds. Dicotyledonous weeds include, but are not limited to, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera.

In addition, the weeds of the present invention can include, for example, crop plants that are growing in an undesired location. For example, a volunteer maize plant that is in a field that predominantly comprises soybean plants can be considered a weed, if the maize plant is undesired in the field of soybean plants.

The articles “a” and “an” are used herein to refer to one or more than one (i.e., to at least one) of the grammatical object of the article. By way of example, “an element” means one or more elements.

As used herein, the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The following examples are offered by way of illustration and not by way of limitation.

### Example 1

### Mutagenesis of Helianthus annuus Line BTK47 and Selection of Imidazolinone-Resistant Plants

BTK47, a Helianthus annuus L. breeding line, was chemically mutated as follows. Sixty thousand seeds of the BTK47 line were treated with a solution of ethyl methanesulfonate (EMS) for 15 hours. The treated seeds were then sown under field conditions on Dec. 16, 2002 at Nidera Experimental Station in Venado Tuerto, Santa Fe, Argentina. Approximately 30,000 M₁ plants flowered and were covered with bags in order to self-pollinate each plant. Each plant was harvested and threshed by hand. On May 10, 2003 at Nidera Experimental Station in Formosa, 20 M₂ seeds from each capitulum were sown under greenhouse conditions. Approximately 590,000 plants were sprayed at V2-V4 stage with imazapyr at a rate of 80 g ai/ha. Eight plants survived the herbicide treatment and were self-pollinated and harvested. M₃ seeds from each of these eight plants were evaluated for their resistance to imazapyr. Out of these eight M₃ families, one family (S4897) segregated for herbicide resistance at a ratio of 1 resistant (i.e., conferring tolerance to commercial rates of imidazolinone herbicides): 2 intermediate (i.e., partially resistant): 1 susceptible. Fully fertile resistant plants were self pollinated and harvested in order to propagate the line S4897.

Leaf tissue from the M3 S4897 plants from both the resistant and intermediate classes were used a source of DNA for analysis of the sequence of the AHASL1 gene as described in Example 2.

### Example 2

### PCR Amplification and Sequencing of Sunflower Polynucleotides Encoding Imidazolinone-Resistant and Wild-Type AHASL1 Proteins

The AHASL1 gene was PCR amplified from DNA isolated from M₃ S4897 and BTK47 (wild-type) sunflower plants as two overlapping fragments. PCR amplification was accomplished with Hotstart Taq DNA polymerase and associated reagents (Quiagen Inc, Valencia, Calif., USA; Cat. No. 203205) using standard methods. The PCR primers for the two fragments are set forth in Table 1 and in the Sequence Listing. HA1U409 (SEQ ID NO: 7) is the forward primer for the first fragment and corresponds to base pair 409 of GenBank Accession No. AY124092. HA1L1379 (SEQ ID NO: 8) is the reverse primer for the first fragment and corresponds to base pair 1379 of GenBank Accession No. AY124092. HA1U1313 (SEQ ID NO: 9) is the forward primer for the second fragment and corresponds to base pair 1313 of GenBank Accession No. AY124092. HA1L2131 (SEQ ID NO: 10) is the reverse primer for the second fragment and corresponds to base pair 2131 of GenBank Accession No. AY124092. The primer pair HA1U409-HA1L1379 produced a 970 base pair fragment. The primer pair HA1U1313-HA1L2131 produced an 818 base pair fragment.

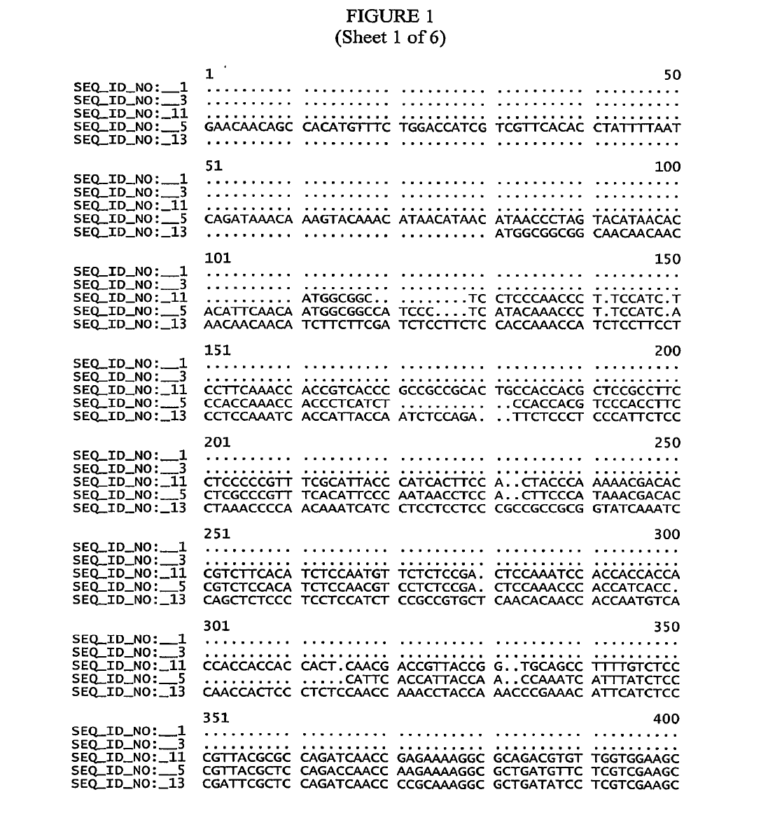

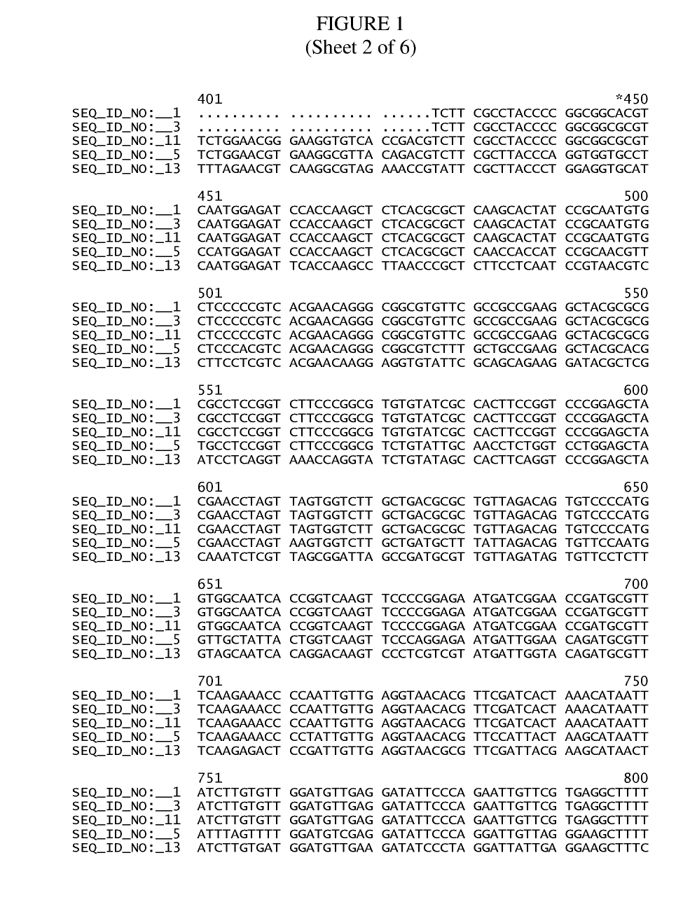

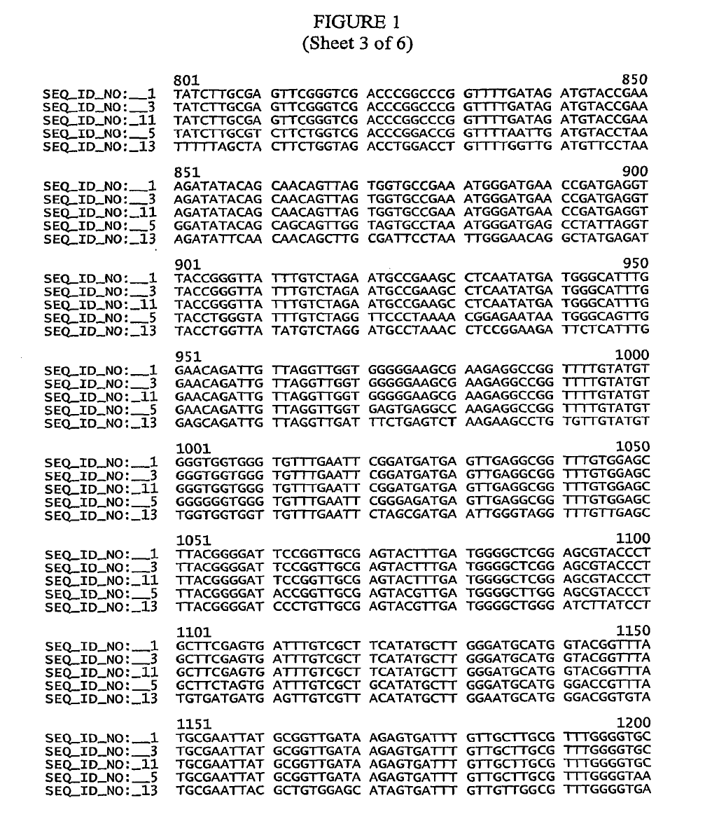

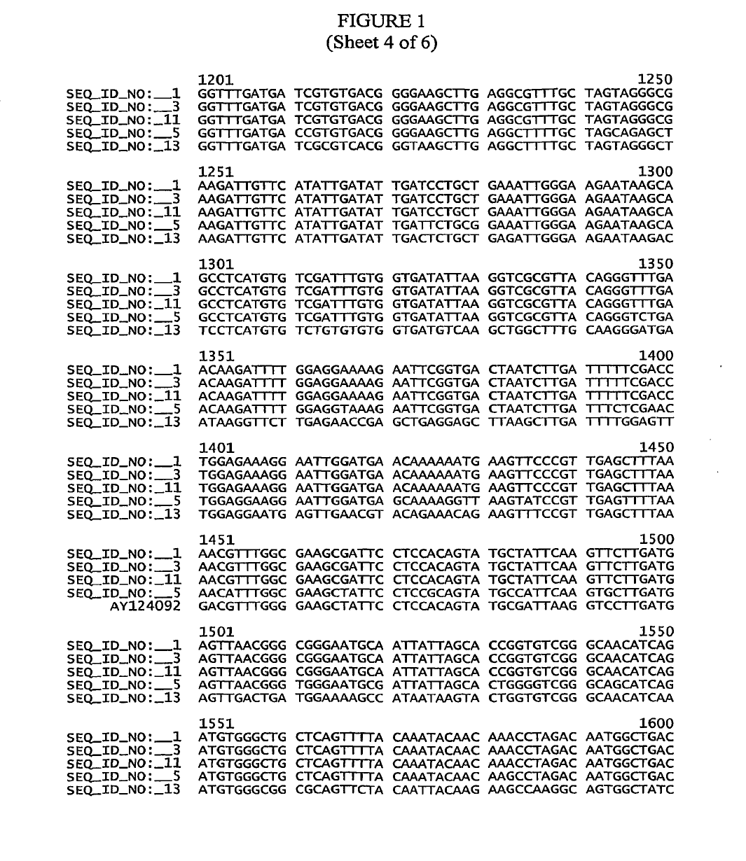

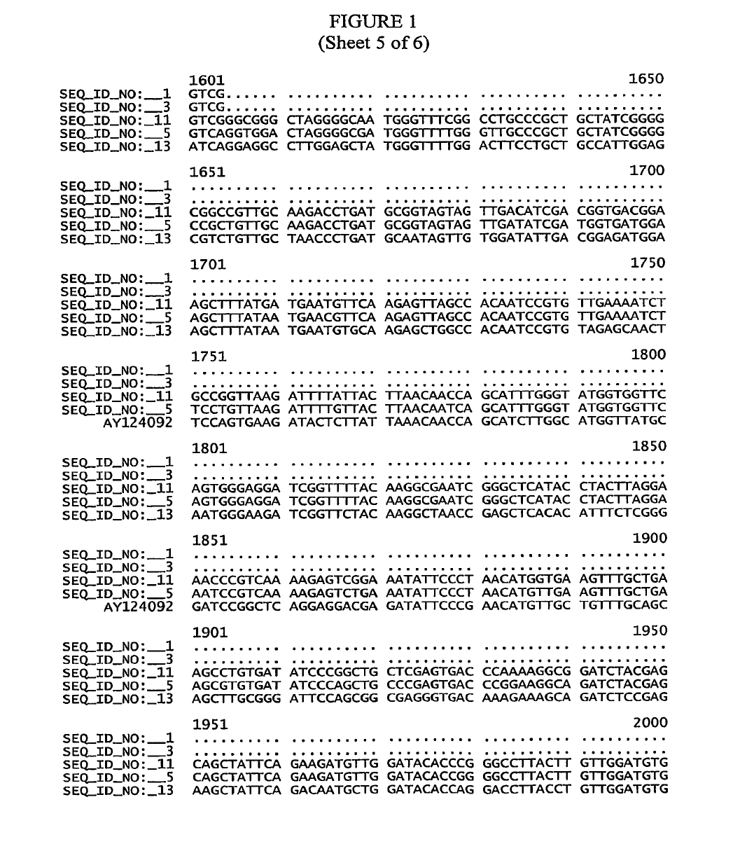

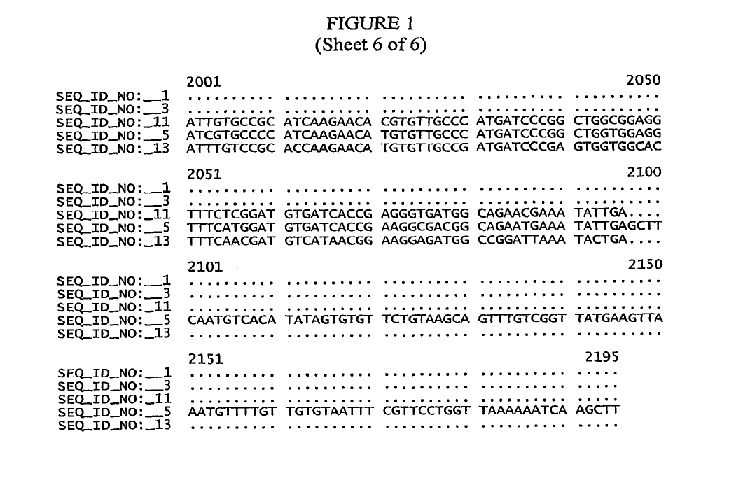

The resulting PCR products were sequenced to produce the AHASL1 sequences for S4897 and BTK47. An alignment of these nucleotide sequences and the nucleotide sequence of the Xanthium sp. ALS gene (GenBank Accession No. U16280; SEQ ID NO: 5) is provided in FIG. 1. The alignment revealed that the AHASL1 gene from S4897 had a single mutation relative to the AHASL1 of BTK47. The site of the mutation is indicated by an asterisk in FIG. 1. This mutation is a G-to-A transition that corresponds to nucleotide 21 of SEQ ID NO: 1.

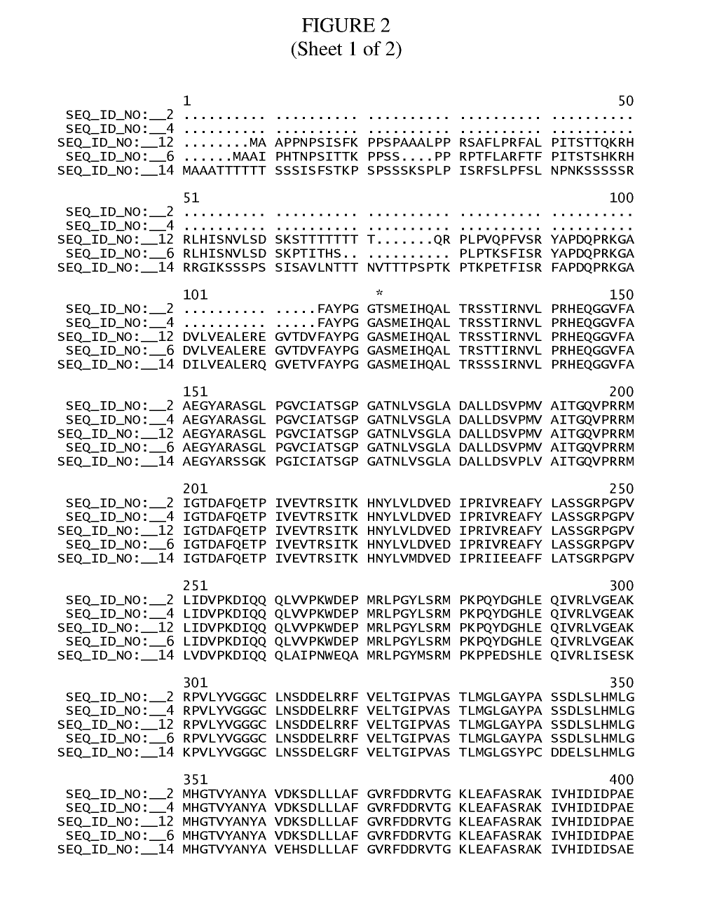

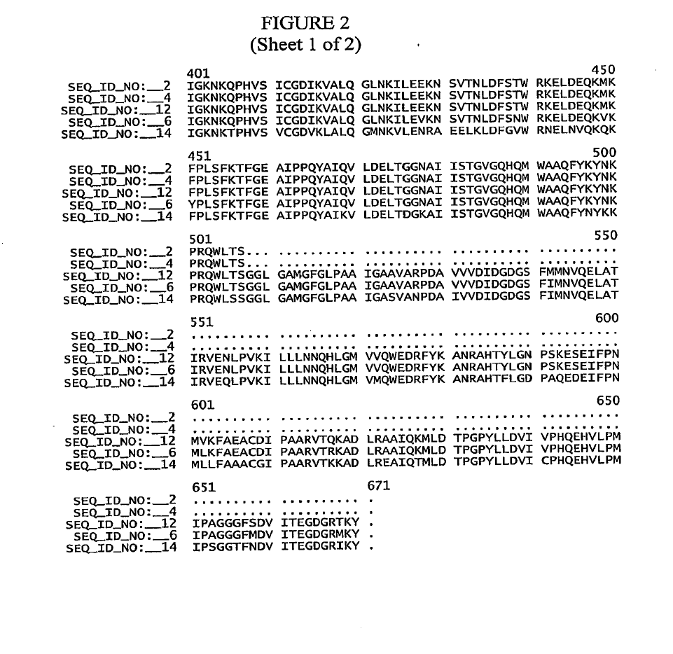

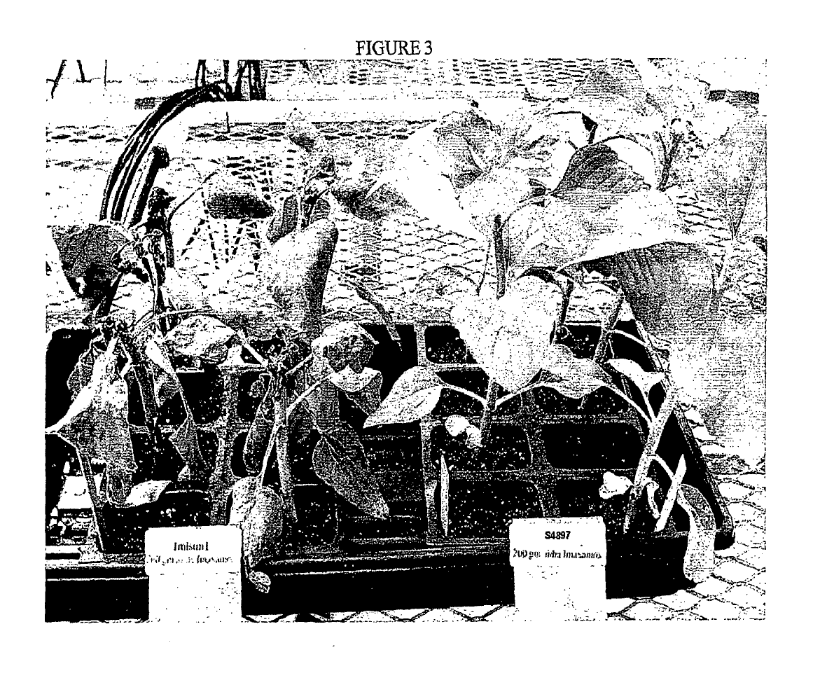

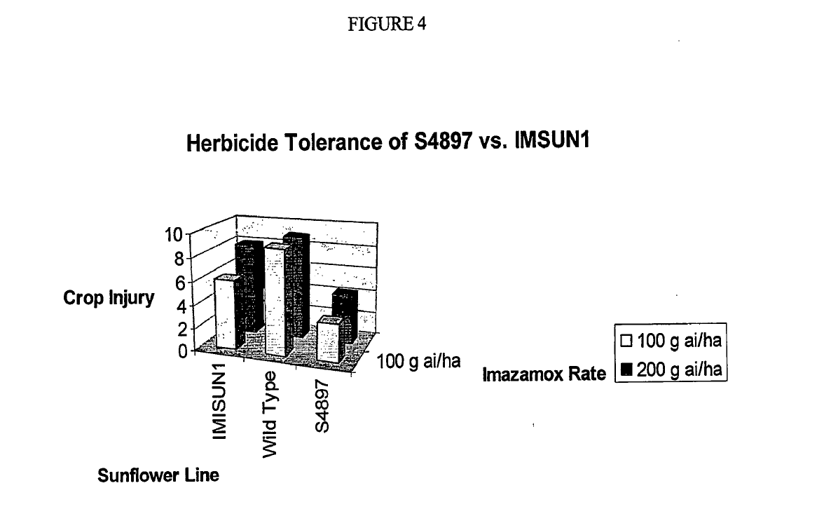

An alignment of the predicted amino acid sequences of the AHASL1 nucleotide sequences of S4897, BTK47, and Xanthium sp. is provided in FIG. 2. Relative to the AHASL1 amino acid sequence of BTK47, the AHASL1 amino acid sequence of S4897 has an alanine-to-threonine substitution at amino acid position 7 (SEQ ID NO: 2). This amino acid position in SEQ ID NO: 2 corresponds to amino acid position 107 in the full-length amino acid sequence encoded by the sunflower AHASL1 nucleotide sequence of GenBank Accession No. AY541451 (SEQ ID NO: 4) and amino acid position 122 in the full-length amino acid sequence encoded by the Arabidopsis thaliana AHASL nucleotide sequence of GenBank Accession No. X51514.

TABLE 1
PCR Primers for Amplifying the Coding Region
of the Sunflower AHASL1 Gene

| Primer Name | Primer Sequence |
|---|---|
| HA1U409 | CAGACGTGTTGGTGGAAGC |
| | (SEQ ID NO: 7) |
| HA1L1379 | CTGTAACGCGACCTTAATATC |
| | (SEQ ID NO: 8) |
| HA1U1313 | TGCTGAAATTGGGAAGAATAAG |
| | (SEQ 1D NO: 9) |
| HA1L2131 | TTTCGTTCTGCCATCACCC |
| | (SEQ ID NO: 10) |

### Example 3

### Production of a Sunflower Line that is Homozygous for the Herbicide Resistance Characteristics of S4897

A sunflower line that is homozygous for the herbicide-resistance trait was produced from S4897 by selfing and screening resistance to imazapyr. This sunflower line and plants thereof were designated as GM40. After confirming the homogeneity of the progeny for herbicide resistance, plants from the GM40 sunflower line were transplanted under greenhouse conditions and self-pollinated for seed production.

### Example 4

### Resistance of S4897 to Imazapic

In field trials in Argentina, S4897 plants were tested for resistance to imazapic. The resistance to imazapic was clearly superior to the IMISUN-1 (Ala₁₉₀-to-Val mutation; equivalent position in Arabidopsis thaliana is 205; see Table 4 below). This trial had single row plots and was treated on a windy day, so the actual herbicide dose reaching the plants was likely less than was applied. Nevertheless, S4897 displayed a superior tolerance to imazapic than the Ala₁₉₀-to-Val, substitution we present no data from this field trial.

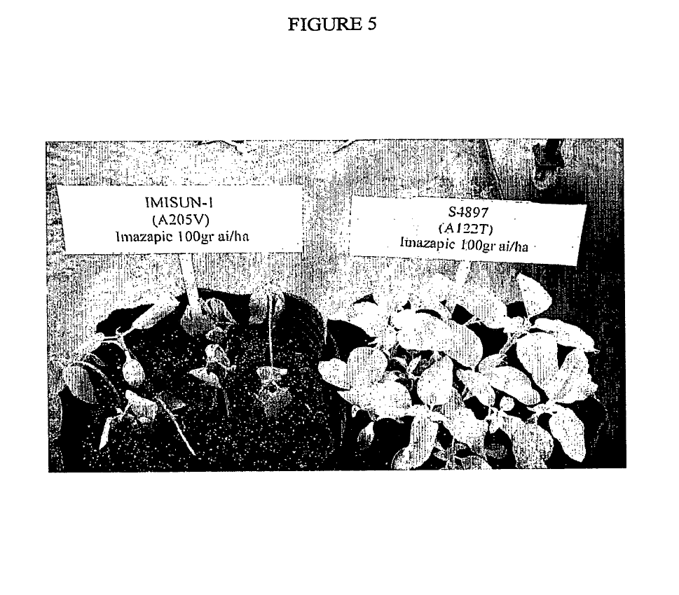

Plants of S4897 and IMISUN-1 were also subjected to imazapic treatment under greenhouse conditions. The photograph in FIG. 5 shows this comparison when the plants were treated with 100 g ai/ha of imazapic.

### Example 5

### Summary of Tolerance and AHAS Activity for Sunflower Line S4897 and Other Clearfield® Varieties of Herbicide-Resistant Sunflower

Methods:

Sunflower lines S4897, Clearfield® sunflower varieties A, B, C and a conventional non-Clearfield variety were sprayed with three rates of imazamox (Raptor™), 100, 200 and 300 gm ai/ha plus 0.5% Sun It II and two rates of imazapyr (Arsenal™) 160 and 360 gm ai/ha plus 0.5% Sun It II when the plants were at the two to three leaf stage. Ratings were taken at 14 days after application (DAT) for injury. Injury was rated on a scale from 0 to 9 where 0=no injury to 9=dead plant. Twelve plants were sprayed per herbicide/rate treatment. Statistical analysis was conducted with STATGRAPHICS Plus 5.0 by using ANOVA and LSD procedures.

AHAS activity analysis was also conducted by selecting actively growing young leaves from plants that were not sprayed with herbicide at approximately four weeks after planting.

Results:

After plants were sprayed, it was realized that a height difference between S4897 and other sunflower varieties would have impacted the actual herbicide dose that was delivered. The spray boom height had been calibrated against S4897 and since the other varieties were taller and closer to the boom they would have received a greater dose of herbicide. Rates were recalibrated for the other varieties to determine the approximate dose they would have received. Assessments were made as presented in Table 2 since it was not possible to make a direct rate comparison due to the effect of the height. For example injury scores were compare for S4897 treated imazamox rate 100 gm ai/ha to the other varieties treated at 75 gm ai/ha. The treatment dose of 300 gm ai/ha for line S4897 was equivalent to the treatment of 200 gm al/ha for the other varieties, which approximately was 300 gm ai/ha.

TABLE 2
Adjustment due/herbicide dose received

| | Actual dose received | | Comparison made | |
|---|---|---|---|---|
| | (gm ai/ha) | | (gm ai/ha) | |
| Herbicide | S4897 | Other varieties | S4897 | Other varieties |
| Imazamox | 50 | 75 | | |
| | 100 | 150 | 100 | 75 |
| | 200 | 300 | 200 | 150 |
| | 300 | 450 | 300 | 300 |
| Imazapyr | 160 | 240 | | |
| | 360 | 540 | 360 | 240 |

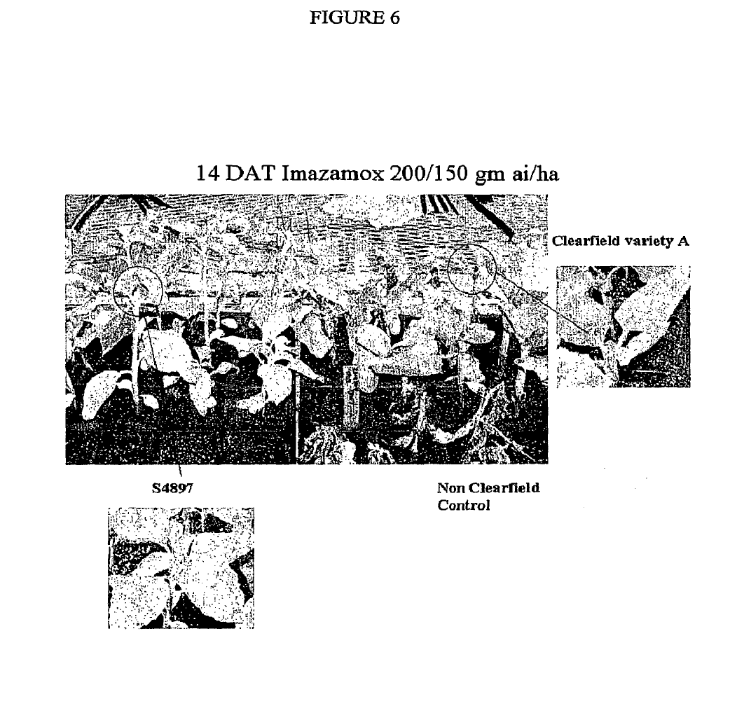

Line S4897 had significantly less injury across all herbicide treatments (Table 3). In fact very little injury was observed at the highest rate of either imazamox or imazapyr. The other varieties showed increasing injury across herbicide rate as would be expected. FIG. 6 shows a comparison of the injury at the 200/150 gm ai/ha for line S4897, Clearfield® variety A, and the non-Clearfield control. The growing tip of S4897 showed little to no injury whereas Clearfield® variety A was significantly in juried and had stopped growing.

TABLE 3
Injury data at 14 DAT for three IMISUN1 lines and S4897 sprayed
with imazamox or imazapyr.

| | Imazamox | | | Imazapyr |
|---|---|---|---|---|
| | (gm ai/ha) | | | (gm ai/ha) |
| Lines | 100/75 | 200/150 | 300 | 360/240 |
| S4897 | 0.5 a | 0.8 a | 0.8 a | 0.8 a |
| Clearfield-Sunflower | 4.3 c | 4.6 c | 6.5 d | 5.0 c |
| variety A | | | | |
| Clearfield-Sunflower | 1.9 b | 4.8 c | 5.3 c | 4.3 b |
| variety B | | | | |
| Clearfield-Sunflower | 1.8 b | 2.9 b | 4.6 b | 4.9 c |
| variety C | | | | |
| Conventional | 7.2 d | 8.2 d | 8.7 e | 8.2 d |
| non-Clearfield | | | | |
| | LSD = 0.7 | LSD = 0.7 | LSD = 0.6 | LSD = 0.7 |

Statistical analysis was conducted with STATGRAPHICS Plus 5.0

Each value is the mean of approximate 12 observation

AHAS activity results also showed less inhibition at the higher concentration of imazamox as compared to a Clearfield® sunflower and conventional non Clearfield variety (FIG. 7). Inhibition by Glean™ was similar across the three sunflower varieties (FIG. 8). Feedback was not presented since the parental background of line S4897 was not available. Since both mutations are in the same AHASL1 locus and all tested varieties were homozygous, there is a qualitative difference in the tolerance conferred by the amino acid substitution in the AHASL1 protein of S4897. Thus, the same amount of AHAS enzyme with this new substitution (i.e., Ala₁₀₇-to-Thr) is capable of catalyzing the formation of more product in the presence of herbicide than does AHAS with the Ala₁₉₀-to-Val substitution. This indicates that AHAS the Ala₁₀₇-to-Thr substitution has superior tolerance to imidazolinone herbicides than AHAS with the Ala₁₉₀-to-Val substitution.

### Example 6

### Herbicide-Resistant Sunflower AHASL Proteins

The present invention discloses both the nucleotide and amino acid sequences for wild-type and herbicide-resistant sunflower AHASL polypeptides. Plants comprising herbicide-resistant AHASL polypeptides have been previously identified, and a number of conserved regions of AHASL polypeptides that are the sites of amino acid substitutions that confer herbicide resistance have been described. See, Devine and Eberlein (1997) “Physiological, biochemical and molecular aspects of herbicide resistance based on altered target sites”. In: Herbicide Activity: Toxicology, Biochemistry and Molecular Biology, Roe et al. (eds.), pp. 159-185, IOS Press, Amsterdam; and Devine and Shukla, (2000) Crop Protection 19:881-889.

Using the AHASL sequences of the invention and methods known to those of ordinary skill in art, one can produce additional polynucleotides encoding herbicide-resistant AHASL polypeptides having one, two, three, or more amino acid substitutions at the identified sites in these conserved regions. Table 4 provides the conserved regions of AHASL proteins, the amino acid substitutions known to confer herbicide resistance within these conserved regions, and the corresponding amino acids in the sunflower AHASL1 protein set forth in SEQ ID NO: 4.

TABLE 4
Mutations in conserved regions of AHASL1 polypeptides known to confer
herbicide-resistance and their equivalent position in sunflower AHASL1

| | | | Amino acid position |
|---|---|---|---|
| Conserved region¹ | Mutation² | Reference | in sunflower |
| VFAYPGGASMEIHQALTRS³ | Ala₁₂₂ to Thr | Bernasconi et al.⁶ | Ala₁₀₇ |
| | | Wright & Penner¹⁴ | |
| AITGQVPRRMIGT⁴ | Pro₁₉₇ to Ala | Boutsalis et al.⁷ | Pro₁₈₂ |
| | Pro₁₉₇ to Thr | Guttieri et al.⁸ | |
| | Pro₁₉₇ to His | Guttieri et al.⁹ | |
| | Pro₁₉₇ to Leu | Guttieri et al.⁸ | |
| | | Kolkman et al.¹⁵ | |
| | Pro₁₉₇ to Arg | Guttieri et al.⁸ | |
| | Pro₁₉₇ to Ile | Boutsalis et al.⁷ | |
| | Pro₁₉₇ to Gln | Guttieri et al.⁸ | |
| | Pro₁₉₇ to Ser | Guttieri et al.⁸ | |
| AFQETP⁴ | Ala₂₀₅ to Asp | Hartnett et al.¹⁰ | Ala₁₉₀ |
| | Ala₂₀₅ to Val | Simpson¹¹ | |
| | | Kolkman et al.¹⁵ | |
| | | White et al.¹⁶ | |
| QWED⁴ | Trp₅₇₄ to Leu | Boutsalis et al.⁷ | Trp₅₅₉ |
| IPSGG⁵ | Ser₆₅₃ to Asn | Devine & | Ala₆₃₈ |
| | | Eberlein¹³ | |
| | Ser₆₅₃ to Thr | Chang & | |
| | Ser₆₅₃ to Phe | Duggleby¹⁷ | |

¹Conserved regions from Devine and Eberlein (1997) “Physiological, biochemical and molecular aspects of herbicide resistance based on altered target sites”. In: Herbicide Activity: Toxicology, Biochemistry and Molecular Biology, Roe et al. (eds.), pp. 159-185, IOS Press, Amsterdam and Devine and Shukla, (2000) Crop Protection 19: 881-889.
²Amino acid numbering corresponds to the amino acid sequence of the Arabidopsis thaliana AHASL1 polypeptide.
³The sunflower AHASL1 amino acid sequences set forth in SEQ ID NOS: 2 and 4 are not full length and begin with the amino acid sequences FAYPGGASMEIHQALTRS and FAYPGGTSMEIHQALTRS, respectively.
⁴The sunflower AHASL amino acid sequences set forth in SEQ ID NOS: 2 and 4 possess this conserved region.
⁵The region of the sunflower AHASL1 (GenBank Accession No. AY541451) corresponding to this conserved region has the sequence IPAGG.
⁶Bernasconi et al. (1995) J. Biol. Chem. 270(29): 17381-17385.
⁷Boutsalis et al. (1999) Pestic. Sci. 55: 507-516.
⁸Guttieri et al. (1995) Weed Sci. 43: 143-178.
⁹Guttieri et al. (1992) Weed Sci. 40: 670-678.
¹⁰Hartnett et al. (1990) “Herbicide-resistant plants carrying mutated acetolactate synthase genes,” In: Managing Resistance to Agrochemicals: Fundamental Research to Practical Strategies, Green et al. (eds.), American Chemical Soc. Symp., Series No. 421, Washington, DC, USA
¹¹Simpson (1998) Down to Earth 53(1): 26-35.
¹²Bruniard (2001) Inheritance of imidazolinone resistance, characterization of cross-resistance pattern, and identification of molecular markers in sunflower (Helianthus annuus L.). Ph.D. Thesis, North Dakota State University, Fargo, ND, USA, pp 1-78.
¹³Devine and Eberlein (1997) “Physiological, biochemical and molecular aspects of herbicide resistance based on altered target sites”. In: Herbicide Activity: Toxicology, Biochemistry and Molecular Biology, Roe et al. (eds.), pp. 159-185, IOS Press, Amsterdam
¹⁴Wright and Penner (1998) Theor. Appl. Genet. 96: 612-620.
¹⁵Kolkman et al. (2004) Theor. Appl. Genet. 109: 1147-1159.
¹⁶White et al. (2003) Weed Sci. 51: 845-853.
¹⁷Chang and Duggleby (1998) Biochem J. 333: 765-777.

### Example 7

### Production of a GM1606 Sunflower Line

A second herbicide-resistant sunflower line was produced by the mutagenesis of sunflower seeds that are wild-type with respect to herbicide resistant by a method essentially as described above in Example 1. The new line and plants of that line are referred to herein as GM1606. The GM1606 sunflower line comprises the same mutation in the AHASL1 gene as in the S4897 sunflower line. This mutation in GM1606 is a G-to-A transition that corresponds to nucleotide 21 of SEQ ID NO: 1, Such a mutation gives rise an alanine-to-threonine substitution at amino acid position 7 (SEQ ID NO: 2). This amino acid position in SEQ ID NO: 2 corresponds to amino acid position 107 in the full-length amino acid sequence encoded by the sunflower AHASL1 nucleotide sequence of GenBank Accession No. AY541451 (SEQ ID NO: 4) and amino acid position 122 in the full-length amino acid sequence encoded by the Arabidopsis thaliana AHASL nucleotide sequence of GenBank Accession No. X51514.

### Example 8

### Response of Mutant Events A122T and A205V to Imazapir

A greenhouse study was conducted to quantify and contrast the imazapir sensitivity of the mutants A122T and A205V in different genetic backgrounds at the whole plant level in sunflower, Seeds of the different sunflower lines that were used in this study were obtained under field conditions. The lines used in the study are listed in Table 5.

TABLE 5
Sunflower Materials

| Hybrid/Line Code | Type of Material | Mutation Event |
|---|---|---|
| TH1 | Hybrid | A205V |
| TH9 | Hybrid | A205V |
| TH10 | Restorer Line | A205V |
| GIM 5-7 | Manteiner Line | A205V |
| IB920 | Manteiner Line | A205V |
| IR79 | Restorer Line | A205V |
| TH6 | Hybrid | A122T |
| TH11 | Hybrid | A122T |
| TH12 | Restorer Line | A122T |
| GM40 | Manteiner Line | A122T |
| GM1606 | Manteiner Line | A122T |
| GIM 5-6 | Restorer Line | A122T |
| TH13 | Manteiner Line | Wild Type |

### Methods

Seeds were sown in Petri dishes and, after germination, plantlets were transplanted to pots of 10 cm of diameter in a potting media consisting of equal parts of vermiculite, soil and sand. Plants were grown in a greenhouse under natural light conditions supplemented with 400 W sodium halide lamps to provide a 16 hrs daylength. Day/night temperatures were 25 and 20° C., respectively. At the V2 stage 10 plants of each genotype were randomly assigned to each treatment consisting of seven imazapir doses (0, 40, 80, 160, 240, 320, 400 and 480 g ai/ha), and a zero-time biomass determination. Experiment was arranged as a randomized block design with a full factorial (sunflower line×treatment) arrangement of treatments and 10 replications.

On the day of herbicide application ten plants of each genotype were cut at the cotyledonal node and dried at 60° C. for 48 hrs zero-time for dried weight determination. The rest of the plants were maintained for 10 days after imazapir treatment (DAT) and their height and root and above ground dry biomass were recorded. Height was determined as the distance between the cotyledonal node and the apex of each plant. For root biomass determination, each plant was taken from the pot and the potting media was washed out from the roots. Above ground biomass data from each line were converted to biomass accumulation after application by subtracting the appropriate average zero-time biomass from each sample. Dry biomass data were converted to percentages of the untreated control plants within each line to allow direct comparisons between groups.

### Results

### 1. Height

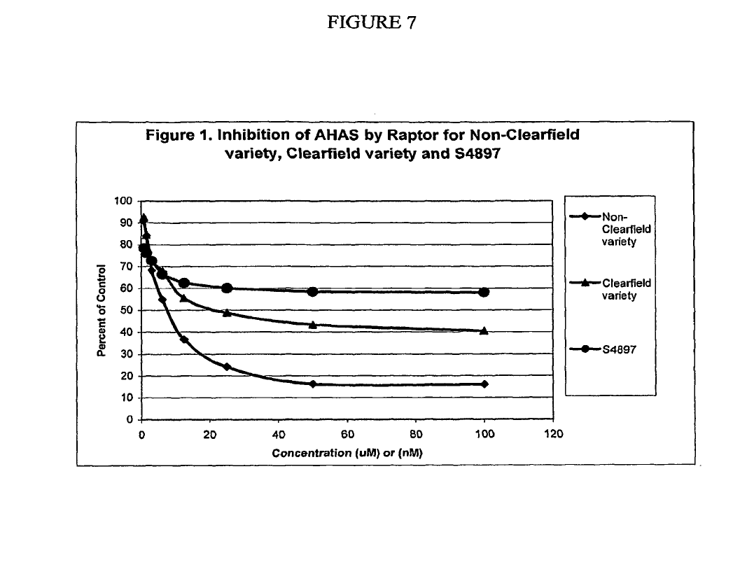

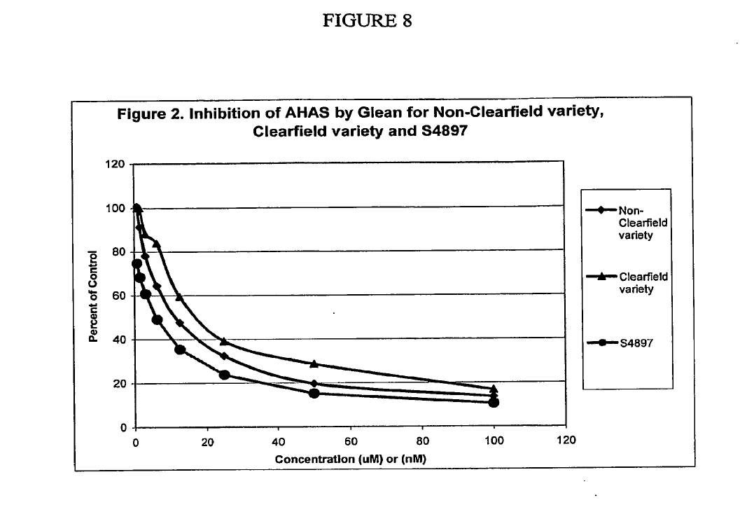

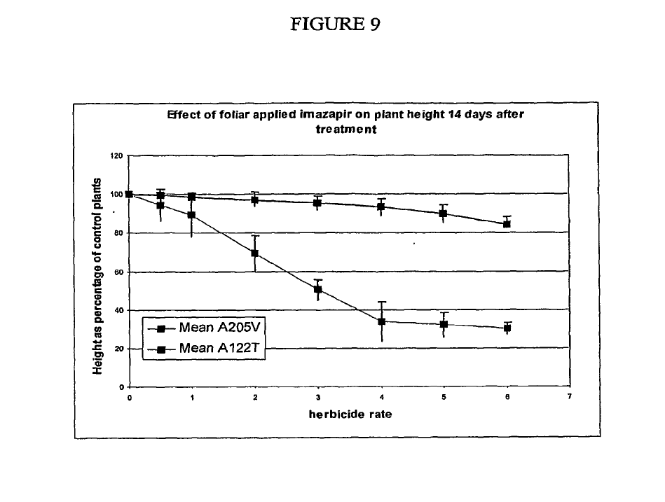

Height of the sunflower lines carrying the A205V mutation did not differ from the untreated controls when a rate of 0.5× or 1× of imazapir was applied. From 2× to 6×, these lines showed a significant reduction in height which reached 68.9%+/−3.1 of the untreated controls (Table 6 and FIG. 9). In contrast, sunflower lines carrying the A122T mutation showed a lesser height reduction (from 0.6 to 15.8% of the untreated controls for 0.5× and 6× rate of imazapir, respectively). Both groups of lines showed a significative difference between them for their response to an increase in herbicide rate from 2× to 6× (Table 6 and FIG. 9).

### 2. Phytotoxicity Index

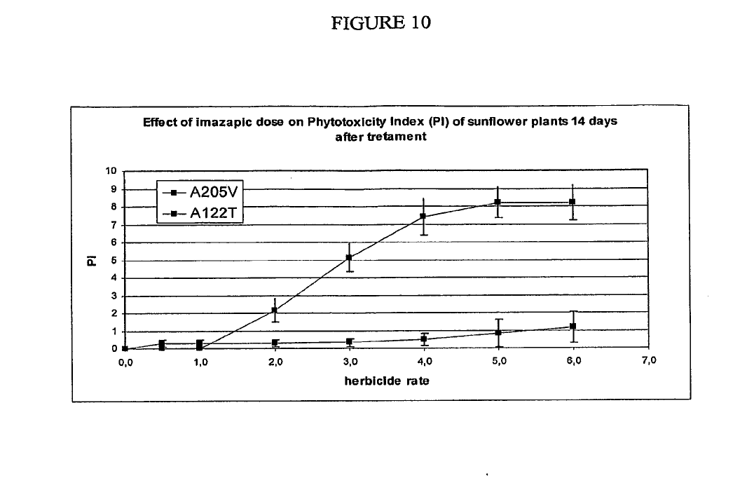

Both mutants showed great differences in their response to the increase in herbicide rate from 0.5× to 6× (FIG. 10). Sunflower lines carrying the A122T mutation showed a slightly reduction in leaf size and lighter green color than the control plants as the herbicide rate increase (Table 7). In contrast, plants carrying the A205V mutation did not show any injury at 0.5× or 1× of herbicide rate, but the level of injury (yellowish, leaf deformation and leaf necrosis) increased quickly from 2× to 6× (Table 7). Both mutants differed between them significantly for the phytotoxicity index from 2× to 6× (Table 7).

### 3. Above Ground Dry Weight Biomass

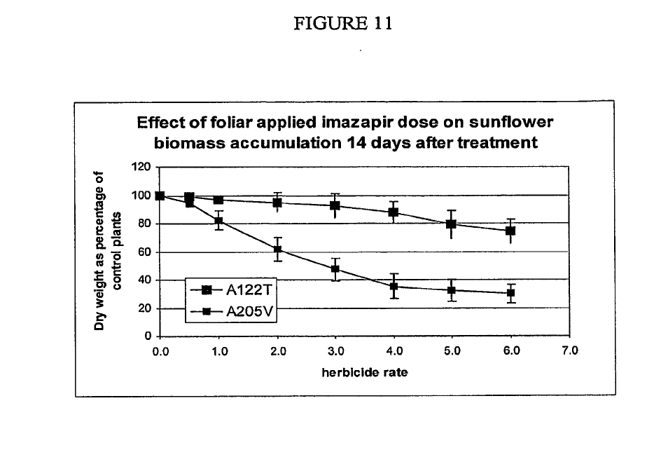

Dose response curves for dry weight of mutants A122T and A205V are shown in FIG. 11. Biomass weight of event A122T was reduced with respect to control plants at 4×, 5× and 6× rates, and this reduction reached 25% for the higher dose. Meanwhile, dry weight of event A205V was reduced with respect to the control plants from 0.5× (40 gai./ha) to 6×. Both mutants showed significant differences between them with respect to this variable from 0.5× to 6× (Table 8). The same trends were obtained for dry matter accumulation (not shown) but without the confounding effects of the initial differences among genotypes for their zero-time dry weight.

### 4. Root Biomass

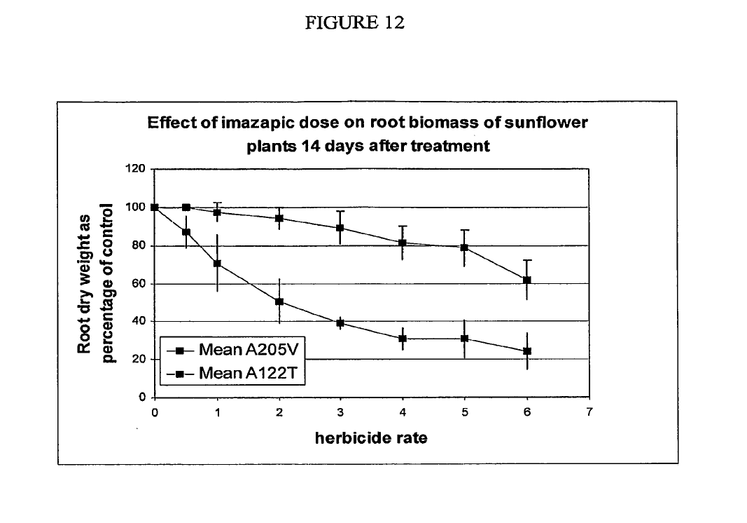

As the doses of imazapir increased, root dry biomass of both mutants were reduced with respect to control plants, but the rate of reduction was very different between A122T and A205V (FIG. 12). In fact, A205V showed a significant reduction in dry weight root biomass from 12.8% at 0.5× (40 g.a.i/ha) to 75.6% at 6× (Table 9). In contrast, A122T carriers showed a significative decrease in root weight biomass from 3× to 6×, and at the higher dose the reduction reached 38.3% (Table 9). Both mutants showed significant differences between them in their root dry weight response to herbicides rates from 0.5× to 6× (Table 9 and FIG. 12).

TABLE 6
Effect of different doses of imazapir on plant height 14 days after treatment for six sunflower
genotypes carrying the A205V mutation event and six genotypes carrying the A122T mutation event.

| | A205V | | | | | | | | Dif with | | A122T | | |
|---|---|---|---|---|---|---|---|---|---|---|---|---|---|
| Dose | TH1 | TH9 | TH10 | GIM5-7 | IB920 | IR79 | Mean | SD | Control | P-value | TH8 | TH11 | TH12 |
| 0 | 100 | 100 | 100 | 100 | 100 | 100 | 100 | 0.0 | — | — | 100 | 100 | 100 |
| 0.5 | 99.5 | 100.0 | 99.2 | 79.1 | 96.2 | 92.8 | 94.4 | 8.0 | 5.6 | 0.15070 | 99.2 | 100.4 | 100.0 |
| 1 | 99.5 | 100.0 | 98.1 | 76.7 | 80.0 | 80.9 | 89.2 | 11.1 | 10.8 | 0.06194 | 98.6 | 99.9 | 100.0 |
| 2 | 78.6 | 78.9 | 63.6 | 57.7 | 61.5 | 74.9 | 69.2 | 9.4 | 30.8 | 0.00048 | 100.3 | 92.0 | 101.8 |
| 3 | 48.4 | 50.0 | 51.9 | 55.3 | 40.9 | 55.7 | 50.4 | 5.5 | 49.6 | 0.00000 | 99.6 | 90.5 | 97.0 |
| 4 | 28.9 | 38.1 | 27.5 | 27.3 | 27.9 | 52.8 | 33.7 | 10.2 | 66.3 | 0.00002 | 101.0 | 90.8 | 92.1 |
| 5 | 25.3 | 31.8 | 26.0 | 37.2 | 30.0 | 41.7 | 32.0 | 6.4 | 68.0 | 0.00000 | 87.0 | 84.4 | 84.8 |
| 6 | 27.1 | 34.7 | 29.3 | 30.0 | 27.1 | 33.2 | 30.2 | 3.1 | 69.8 | 0.00000 | 79.6 | 84.4 | 79.8 |

| | A122T | | | | | Dif with | | Dif | |
|---|---|---|---|---|---|---|---|---|---|
| Dose | GM40 | GIM5-6 | GM1606 | Mean | SD | Control | P-value | A122T − A205V | P-value |
| 0 | 100 | 100 | 100 | 100 | 0 | — | — | | |
| 0.5 | 99.6 | 100.0 | 97.0 | 99.4 | 1.2 | 0.6 | 0.25170 | 4.9 | 0.19611 |
| 1 | 97.4 | 100.0 | 95.7 | 98.6 | 1.8 | 1.4 | 0.10824 | 9.4 | 0.09183 |
| 2 | 95.2 | 99.1 | 95.2 | 97.3 | 3.7 | 2.7 | 0.13184 | 28.0 | 0.00033 |
| 3 | 93.0 | 98.1 | 95.7 | 95.6 | 3.4 | 4.4 | 0.02466 | 45.3 | 0.00000 |
| 4 | 93.0 | 91.0 | 94.1 | 93.7 | 3.8 | 6.3 | 0.00970 | 59.9 | 0.00001 |
| 5 | 93.9 | 94.3 | 94.3 | 89.8 | 4.9 | 10.2 | 0.00371 | 57.8 | 0.00000 |
| 6 | 84.3 | 85.8 | 91.4 | 84.2 | 4.4 | 15.8 | 0.00031 | 54.0 | 0.00000 |

TABLE 7
Effect of different doses of imazapir on Phytotoxicity Index 14 days after treatment for three sunflower
genotypes carrying the A205V mutation event and three genotypes carrying the A122T mutation event.

| | A205V | | | | | | A122T | | | | | | Dif | |
|---|---|---|---|---|---|---|---|---|---|---|---|---|---|---|
| Dose | TH1 | TH9 | TH10 | Mean | SD | P-value | TH6 | TH11 | TH12 | Mean | SD | P-value | A205V − A122T | P-value |
| 0 | 0 | 0 | 0 | 0 | 0 | | 0 | 0 | 0 | 0 | 0 | | | |
| 0.5 | 0 | 0 | 0 | 0 | 0 | ns | 0.5 | 0.4 | 0.0 | 0.3 | 0.3 | 0.19171 | −0.29 | 0.19171 |
| 1 | 0 | 0 | 0 | 0 | 0 | ns | 0.5 | 0.4 | 0.0 | 0.3 | 0.3 | 0.19461 | −0.29 | 0.19461 |
| 2 | 1.8 | 1.6 | 3.1 | 2.2 | 0.8 | 0.04648 | 0.5 | 0.4 | 0.0 | 0.3 | 0.3 | 0.18981 | 1.87 | 0.05030 |
| 3 | 6.4 | 5.1 | 3.9 | 5.1 | 1.2 | 0.01793 | 0.5 | 0.5 | 0.0 | 0.3 | 0.3 | 0.18350 | 4.81 | 0.01622 |
| 4 | 8.0 | 8.4 | 5.9 | 7.4 | 1.3 | 0.01084 | 0.5 | 1.0 | 0.0 | 0.5 | 0.5 | 0.22540 | 6.92 | 0.00657 |
| 5 | 8.9 | 8.9 | 6.9 | 8.2 | 1.1 | 0.00601 | 0.5 | 2.0 | 0.0 | 0.8 | 1.0 | 0.29986 | 7.38 | 0.00111 |
| 6 | 9.0 | 8.9 | 6.7 | 8.2 | 1.3 | 0.00799 | 0.5 | 2.5 | 0.5 | 1.2 | 1.2 | 0.22222 | 7.03 | 0.00219 |

TABLE 8
Effect of different doses of imazapir on biomass accumulation 14 days after treatment for six sunflower
genotypes carrying the A205V mutation event and six genotypes carrying the A122T mutation event

| | A205V | | | | | | | | Dif with | | A122T | | |
|---|---|---|---|---|---|---|---|---|---|---|---|---|---|
| Dose | TH1 | TH9 | TH10 | GIM5-7 | IB920 | IR79 | Mean | SD | control | P-value | TH6 | TH11 | TH12 |
| 0.0 | 100 | 100 | 100 | 100 | 100 | 100 | 100 | 0.0 | — | — | 100 | 100 | 100 |
| 0.5 | 95.0 | 91.7 | 99.2 | 92.9 | 93.9 | 94.4 | 94.5 | 2.6 | 5.5 | 0.00338 | 100 | 96.6 | 100.0 |
| 1.0 | 89.6 | 81.7 | 85.0 | 75.1 | 87.9 | 74.0 | 82.2 | 6.5 | 17.8 | 0.00116 | 97.2 | 93.9 | 99.1 |
| 2.0 | 75.5 | 54.7 | 58.1 | 55.1 | 58.8 | 67.7 | 61.6 | 8.2 | 38.4 | 0.00009 | 97.9 | 81.6 | 97.0 |
| 3.0 | 60.4 | 35.7 | 48.1 | 45.6 | 46.4 | 49.1 | 47.6 | 7.9 | 52.4 | 0.00002 | 98.2 | 75.8 | 96.1 |
| 4.0 | 46.5 | 25.3 | 28.8 | 31.8 | 35.5 | 44.5 | 35.4 | 8.5 | 64.6 | 0.00001 | 97.8 | 75.0 | 84.3 |
| 5.0 | 38.9 | 19.8 | 27.4 | 34.2 | 33.9 | 40.1 | 32.4 | 7.6 | 67.6 | 0.00000 | 85.1 | 60.1 | 77.5 |
| 6.0 | 33.9 | 19.5 | 24.9 | 36.8 | 29.4 | 35.3 | 30.0 | 6.7 | 70.0 | 0.00000 | 79.5 | 59.6 | 70.7 |

| | A122T | | | | | Dif with | | Dif | |
|---|---|---|---|---|---|---|---|---|---|
| Dose | GM40 | GIM5-6 | GM1606 | Mean | SD | control | P-value | A122T − A205V | P-value |
| 0.0 | 100 | 100 | 100 | 100 | 0 | — | — | — | — |
| 0.5 | 96.5 | 100.0 | 100.0 | 98.9 | 1.8 | 1.1 | 0.18453 | 4.3 | 0.00823 |
| 1.0 | 94.1 | 96.4 | 100.0 | 96.8 | 2.5 | 3.2 | 0.02634 | 14.5 | 0.00182 |
| 2.0 | 97.6 | 97.7 | 97.6 | 94.9 | 6.5 | 5.1 | 0.11375 | 33.2 | 0.00002 |
| 3.0 | 95.8 | 95.8 | 95.4 | 92.8 | 8.4 | 7.2 | 0.09241 | 45.3 | 0.00000 |
| 4.0 | 88.3 | 90.8 | 90.5 | 87.8 | 7.6 | 12.2 | 0.01127 | 52.4 | 0.00000 |
| 5.0 | 85.6 | 81.9 | 85.3 | 79.3 | 9.9 | 20.7 | 0.00362 | 46.9 | 0.00001 |
| 6.0 | 76.8 | 78.0 | 83.0 | 74.6 | 8.4 | 25.4 | 0.00070 | 44.6 | 0.00000 |

TABLE 9
Effect of different doses of imazapir on root dry weight 14 days after treatment for six sunflower
genotypes carrying the A205V mutation event and six genotypes carrying the A122T mutation event

| | A205V | | | | | | | | Dif with | | A122T | | |
|---|---|---|---|---|---|---|---|---|---|---|---|---|---|
| Dose | TH1 | TH9 | TH10 | GIM5-7 | IB920 | IR79 | Mean | SD | control | P-value | TH6 | TH11 | TH12 |
| 0 | 100 | 100 | 100 | 100 | 100 | 100 | 100 | 0.0 | 0.0 | — | 100 | 100 | 100 |
| 0.5 | 79.4 | 81.5 | 90.3 | 79.2 | 94.0 | 98.6 | 87.2 | 8.3 | 12.8 | 0.01281 | 100 | 100 | 99.4 |
| 1 | 70.7 | 84.9 | 69.9 | 56.3 | 89.6 | 53.4 | 70.8 | 14.6 | 29.2 | 0.00447 | 98.1 | 99.0 | 100 |
| 2 | 69.6 | 58.8 | 41.3 | 40.6 | 50.7 | 42.5 | 50.6 | 11.7 | 49.4 | 0.00014 | 96.2 | 86.1 | 100 |
| 3 | 42.3 | 34.1 | 42.8 | 40.2 | 37.3 | 38.4 | 39.2 | 3.3 | 60.8 | 0.00000 | 79.3 | 81.5 | 100.0 |
| 4 | 38.6 | 27.3 | 22.3 | 34.0 | 32.8 | 31.5 | 31.1 | 5.7 | 68.9 | 0.00000 | 78.4 | 72.6 | 74.5 |
| 5 | 34.7 | 29.9 | 22.7 | 48.5 | 25.4 | 23.3 | 30.7 | 9.8 | 69.3 | 0.00001 | 85.8 | 68.9 | 65.0 |
| 6 | 23.1 | 22.1 | 16.4 | 43.1 | 19.4 | 21.9 | 24.4 | 9.5 | 75.6 | 0.00001 | 57.3 | 45.3 | 57.8 |

| | A122T | | | | | Dif with | | Dif | |
|---|---|---|---|---|---|---|---|---|---|
| Dose | GM40 | GIM5-6 | GM1606 | Mean | SD | control | P-value | A122T − A205V | P-value |
| 0 | 100 | 100 | 100 | 100 | 0.0 | 0.0 | | 0.0 | — |
| 0.5 | 100 | 100 | 100 | 99.9 | 0.2 | 0.1 | 0.36322 | 12.7 | 0.01314 |
| 1 | 100 | 88.0 | 100.0 | 97.5 | 4.7 | 2.5 | 0.25371 | 26.7 | 0.00520 |
| 2 | 100 | 90.0 | 92.5 | 94.1 | 5.6 | 5.9 | 0.05070 | 43.6 | 0.00006 |
| 3 | 92.2 | 98.0 | 85.2 | 89.4 | 8.7 | 10.6 | 0.02997 | 50.2 | 0.00001 |
| 4 | 96.1 | 80.0 | 87.0 | 81.4 | 8.7 | 18.6 | 0.00346 | 50.3 | 0.00000 |
| 5 | 84.2 | 80.0 | 88.1 | 78.7 | 9.5 | 21.3 | 0.00273 | 47.9 | 0.00001 |
| 6 | 74.3 | 65.8 | 69.5 | 61.7 | 10.4 | 38.3 | 0.00028 | 37.3 | 0.00007 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1-15. (canceled)

16. A method for controlling weeds in a field of a sunflower plant by the application of an herbicide without significantly inhibiting the growth of said plant, said method comprising:
(a) growing in the field:
(i) a sunflower plant or seed of line GM40 or GM1606, a representative sample of seed of each line having been deposited under ATCC Patent Deposit Number PTA-6716 and, ATCC Patent Deposit Number PTA-7606, respectively;
(ii) a sunflower plant or seed that is a progeny of line or GM1606, a representative sample of seed of each line having been deposited under ATCC Patent Deposit Number PTA-6716 and ATCC Patent Deposit Number PTA-7606, respectively, said sunflower plant or seed comprising the herbicide-resistance characteristics of line GM40 or GM1606, a representative sample of seed of each line having been deposited under ATCC Patent Deposit Number PTA-6716 and ATCC Patent Deposit Number PTA-7606, respectively;
(iii) a sunflower plant or seed that is a mutant, recombinant, or a genetically engineered derivative of line GM40 or GM1606, a representative sample of seed of each line having been deposited under ATCC Patent Deposit Number PTA-6716 and ATCC Patent Deposit Number PTA-7606, respectively; or
(iv) a sunflower plant or seed that is a progeny of any of the sunflower plants of (i)-(iii), said sunflower plant comprising the herbicide-resistance characteristics of line GM40 or GM1606, a representative sample of seed of each line having been deposited under ATCC Patent Deposit Number PTA-6716 and ATCC Patent Deposit Number PTA-7606, respectively; and
(b) applying an effective amount of an imidazolinone herbicide, or a mixture of an imidazolinone herbicide, a sulfonylurea herbicide, and/or a triazolopyrimidine herbicide:
(i) to the field, followed by planting of said plant or seed in therein;
(ii) to the field, during or after planting of said seed therein;
(iii) to the plant in said field and to weeds in the vicinity of the plant;
(iv) to said seed, followed by planting of said seed in the field; or
(v) to a plant by the seed after it has been planted in the field, and to weeds in the vicinity of the plant;
wherein said effective amount of said herbicide would significantly inhibit the growth of a corresponding wild-type variety of the plant;
thereby controlling weeds.

17. The method of claim 16, wherein said imidazolinone herbicide comprises: 2-(4-isopropyl-4-methyl-5-oxo-2-imidazolin-2-yl)-nicotinic acid, 2-(4-isopropyl-4-methyl-5-oxo-2-imidazolin-2-yl)-3-quinolinecarboxylic acid, 5-ethyl-2-(4-isopropyl-4-methyl-5-oxo-2-imidazolin-2-yl)-nicotinic acid 2-(4-isopropyl-4-methyl-5-oxo-2-imidazolin-2-yl)-5-(methoxymethyl)-nicotinic acid, 2-(4-isopropyl-4-methyl-5-oxo-2-imidazolin-2-yl)-5-methylnicotinic acid, a mixture of methyl 6-(4-isopropyl-4-methyl-5-oxo-2-imidazolin-2-yl)-m-toluate[H] and methyl 2-(4-isopropyl-4-methyl-5-oxo-2-imidazolin-2-yl)-p-toluate, or a mixture of any of the foregoing.

18. The method of claim 16, wherein said sulfonylurea herbicide comprises: chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfuron, flazasulfuron, imazosulfuron, pyrazosulfuron ethyl, halosulfuron, or a mixture of any of the foregoing.

19-85. (canceled)

86. A method for combating undesired vegetation comprising contacting a seed of a sunflower plant before sowing and/or after pregermination with an AHAS-inhibiting herbicide, wherein said sunflower plant comprises in its genome at least one copy of at least one endogenous gene comprising an acetohydroxyacid synthase large subunit (AHASL) polynucleotide, wherein said AHASL polynucleotide encodes a herbicide-tolerant AHASL1 protein comprising:
(a) the amino acid sequence set forth in SEQ ID NO:2 with a substitution from alanine to threonine at amino acid position 7, corresponding to position 107 of SEQ ID NO:12, said substitution having been obtained by induced mutagenesis, or a variant thereof that further contains at least one member selected from the group consisting of:
(i) a substitution from proline to glutamine or serine at amino acid position 182 or an equivalent position relative to the amino acid sequence set forth in SEQ ID NO:12;
(ii) a substitution from threonine to isoleucine or an amino acid other than threonine at amino acid position 188 or an equivalent position relative to the amino acid sequence set forth in SEQ ID NO:12;
(iii) a substitution from alanine to aspartate or valine at amino acid position 190 or an equivalent position relative to the amino acid sequence set forth in SEQ ID NO:12;
(iv) a substitution from tryptophan to leucine at amino acid position 559 or an equivalent position relative to the amino acid sequence set forth in SEQ ID NO:12; and
(v) a substitution from alanine any one of asparagine, threonine, phenylalanine, or valine at amino acid position 638 or an equivalent position relative to the amino acid sequence set forth in SEQ ID NO:12; or
(b) the amino acid sequence set forth in SEQ ID NO:2 with a substitution from alanine to another amino acid other than alanine at amino acid position 7, corresponding to position 107 of SEQ ID NO:12, said substitution having been obtained by induced mutagenesis, or a variant thereof that further contains at least one member selected from the group consisting of:
(i) a substitution from proline to alanine, threonine, histidine, leucine, arginine, or isoleucine at amino acid position 182 or an equivalent position relative to the amino acid sequence set forth in SEQ ID NO:12, wherein the protein does not comprise a threonine at amino acid position 107 or an equivalent position relative to the amino acid sequence set forth in SEQ ID NO:12;
(ii) a substitution from proline to glutamine or serine at amino acid position 182 or an equivalent position relative to the amino acid sequence set forth in SEQ ID NO:12;
(iii) a substitution from threonine to isoleucine or an amino acid other than threonine at amino acid position 188 or an equivalent position relative to the amino acid sequence set forth in SEQ ID NO:12;
(iv) a substitution from alanine to aspartate or valine at amino acid position 190 or an equivalent position relative to the amino acid sequence set forth in SEQ ID NO:12;
(v) a substitution from tryptophan to leucine at amino acid position 559 or an equivalent position relative to the amino acid sequence set forth in SEQ ID NO:12; and
(vi) a substitution from alanine to any one of asparagine, threonine, phenylalanine, or valine at amino acid position 638 or an equivalent position relative to the amino acid sequence set forth in SEQ ID NO:12; or
(c) a progeny sunflower plant of (a) or (b); and
wherein said plant, or progeny thereof, has increased tolerance to at least one AHAS-inhibiting herbicide as compared to a wild-type variety of the sunflower plant.

87. The method of claim 86, wherein said AHAS-inhibiting herbicide comprises an imidazolinone herbicide.

88. The method of claim 87 wherein said imidazolinone herbicide comprises: 2-(4-isopropyl-4-methyl-5-oxo-2-imidazolin-2-yl)-nicotinic acid, 2-(4-isopropyl-4-methyl-5-oxo-2-imidazolin-2-yl)-3-quinolinecarboxylic acid, 5-ethyl-2-(4-isopropyl-4-methyl-5-oxo-2-imidazolin-2-yl)-nicotinic acid, 2-(4-isopropyl-4-methyl-5-oxo-2-imidazolin-2-yl)-5-(methoxymethyl)-nicotinic acid, 2-(4-isopropyl-4-methyl-5-oxo-2-imidazolin-2-yl)-5-methylnicotinic acid, a mixture of methyl 6-(4-isopropyl-4-methyl-5-oxo-2-imidazolin-2-yl)-m-toluate and methyl 2-(4-isopropyl-4-methyl-5-oxo-2-imidazolin-2-yl)-p-toluate, or a mixture of any of the foregoing.

89. The method of claim 86, wherein said AHAS-inhibiting herbicide comprises a sulfonylurea herbicide.

90. The method of claim 89, wherein said sulfonylurea herbicide comprises: chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfuron, flazasulfuron, imazosulfuron, pyrazosulfuron ethyl, halosulfuron, or a mixture of any of the foregoing.

91. A method for detecting a sunflower AHASL1, said method comprising:
(a) providing or obtaining biological material from a plant or plant part;
(b) performing PCR or hybridization testing of said biological material to determine whether the biological material comprises a polynucleotide comprising:
(a) the nucleotide sequence set forth in SEQ ID NO:1;
(b) a nucleotide sequence encoding the amino acid sequence set forth in SEQ ID NO:2;
(c) a nucleotide sequence having at least 90% sequence identity to the nucleotide sequence set forth in SEQ ID NO:1; or
(d) a nucleotide sequence encoding a protein having at least 90% sequence identity to the amino acid sequence set forth in SEQ ID NO:2; and
(c) identifying, based on the results of step (b), that the plant or plant part of step (a) comprises said polynucleotide.

92. The method of claim 91, wherein said biological material is a plant seed.

93. The method of claim 91, wherein the PCR or hybridization testing comprises the use of a primer or probe comprising any of the polynucleotide sequences of SEQ ID NOs:7, 8, 9, or 10.

94. The method of claim 93, wherein at least one of said primers or probes is attached to a detectable label.
